(19) United States
(12) Patent Application Publication
(10) Pub. No.: US 2020/0046525 A1
(43) Pub. Date: Feb. 13, 2020

(54) FLANGE STENT DEVICE AND MODULAR STENT SYSTEM THEREOF

(71) Applicants: András Kónya, Houston, TX (US); Jeffery J. Sheldon, League City, TX (US)

(72) Inventors: András Kónya, Houston, TX (US); Jeffery J. Sheldon, League City, TX (US)

(21) Appl. No.: 16/530,633

(22) Filed: Aug. 2, 2019

Related U.S. Application Data
(60) Provisional application No. 62/716,141, filed on Aug. 8, 2018.

(51) Int. Cl. A61F 2/88; A61B 17/12; A61F 2/07; A61F 2/90

(52) U.S. Cl. CPC A61F 2/88; A61B 17/12118; A61B 2017/00526; A61F 2/90; A61F 2/07

(57) ABSTRACT

This disclosure provides a self-expandable, woven stent in a variety of anatomical structures, including vessels in the arterial and venous system. The disclosed stent may comprise a body portion with a flange portion at one of its ends. The flange stent may also be a part of a modular stent assembly and/or utilized with other stents that are not flanged. In use, a first stent may be placed in a first vessel and a second stent (which may be a flanged stent) may be placed in a second vessel through a wall of the first stent. The flange stent may be created by bending shape memory wires around a template with a flanged portion or heat treating a substantially straight stent around a template with a flange or forming a stent with a tighter mesh on a portion of the stent over a substantially straight template.

### PRIORITY

This application claims priority to U.S. provisional patent application No. 62/716,141, filed on Aug. 8, 2018, the entire contents of which is incorporated herein by reference.

### BACKGROUND OF THE INVENTION

### Field of the Invention

The present disclosure relates generally to the field of intravascular devices. More particularly, it concerns a flange stent that may be used as a modular stent, and the methods of making the same, and the apparatus and methods for delivery of the same into a living creature.

### Description of the Related Art

In general, a “lesion” is an injury, hurt or wound, and in particular a localized, abnormal structure change in the body, such as diseased tissue. For the purposes of this disclosure, lesions may be aorta-ostial lesions, non aorto-ostial lesions, and/or branch ostial lesions. An “ostial lesion” as used herein is a lesion that involves the very first part of a side vessel branch, which is frequently in continuation with lesions of the main (parent) vessel. Ostial lesions are usually fibrotic, calcified, and relatively rigid. As has been suggested by those of skill in the art, aorto-ostial disease may be resistant to dilation and prone to recoil due to the greater thickness of muscular and elastic tissue in the aortic wall. Thus, ostial lesions are very difficult to treat, and existing stents and procedures are generally ineffective and problematic for various reasons. An ostial lesion requires precise implantation of a stent to prevent adverse clinical outcomes. “Geographic miss” occurs when the ostial side-branch lesion is not fully covered by the stent. Conversely, a stent placed too proximal to the main vessel may complicate future intervention or lead to acute problems in the main branch. Stent sizing is a challenging issue, especially in complex lesions.

FIGS. 1A-1D illustrate a branching anatomic duct with and without stents. In the prior art, treating a lesion with a stent in a branching anatomic duct is a prevalent and particularly difficult geometry to achieve placement of a stent at the exact location of an atherosclerotic blockage. FIG. 1A illustrates an example of a branching anatomic duct 10 with atherosclerotic plaque 16 at ostium 18 of side branch duct 14 of main branch duct 12. Side branch 14 and main branch 12 form an angled relationship which may be a right or acute angle. In the prior art, stents are typically positioned improperly within the ducts. FIGS. 1B-1D illustrate some examples of poorly positioned prior art stents. For example, FIG. 1B illustrates an example of an improper longitudinal positioning of a stent where proximal end 20 of stent 30 may protrude into the lumen of main branch duct 12. The proximal end then becomes a partial obstruction of the lumen that impedes the blood flow both into both side branch 14 and main branch 12. Furthermore, such a positioning poses a potential danger of turbulent circulation, thrombus development, and further complications, such as embolism. FIG. 1C illustrates an example of another misplacement of stent 30. In this example, stent 30 is positioned too deeply in side branch 14, thereby leaving residual untreated atherosclerotic plaque 16A, which can cause a blockage at ostium 18 of side branch 14 thereby blocking the flow into branching duct 10. FIG. 1D illustrates an example of another misplacement of stent 30. In this example, stent 30 is placed in a branching duct 10 where arcuate segment 32 of stent 30 overhangs the ostium 18 and becomes a flow impending partial obstruction. The residual untreated plaque 16A remains immediately adjacent to ostium 18 and also becomes a flow impending partial obstruction.

Stenting of bifurcation lesions is a complex issue, resulting in lower angiographic success rates due to complications like acute vessel closure and a higher risk of restenosis. Coronary bifurcation lesions comprise a significant amount of percutaneous coronary interventions (PCI). However, it is well known that procedural and clinical outcomes associated with coronary bifurcation treatment are suboptimal due to the complexity of anatomy and the dynamic changes that occur during PCI. As is known in the art, a “true” bifurcation lesion involves significant (e.g., greater than 50%) stenosis in both the main branch and the side branch, whereas all other lesion types may be classified as “nontrue.” The treatment for bifurcation lesions is particularly challenging, and treatment with the currently used straight metal stents does not produce optimal results.

There are numerous conventional treatments for bifurcation lesions. For example, FIGS. 2A-2H illustrate various techniques for stenting bifurcation lesions. A lesion may be treated by using a one-stent approach or a two-stent approach. A two-stent approach may be preferred for true bifurcation lesions. In most situations, a stent is provided in the main vessel with provisional stenting on the side branch. FIG. 2A illustrates a typical bifurcation lesion showing main vessel 212 and side branch vessel 214. As illustrated in FIGS. 2B-2H and as is known in the art, typical stenting procedures include stent+PTCA (FIG. 2B), T-stenting (FIG. 2C), reverse T-stenting (FIG. 2D), culotte stenting (FIG. 2E), Y or V stenting (FIG. 2F), crush stenting (FIG. 2G), and simultaneous-kissing stents (FIG. 2H), among others. Element 1 in these figures is a first stent, and element 2 in these figures is a second stent. These techniques/procedures are all problematic, including being tedious and cumbersome, having unpredictable outcomes, resulting in gaps in tissue coverage or excessive metal and injury, being incomplete or excessive coverage, requiring repeated procedures for revascularization, and suffering from high restenosis rates. Each of the embodiments illustrated in FIGS. 2A-2H using conventional stents creates numerous problems, a few of which are described below.

For example, as illustrated in the Stent+PTCA embodiment (FIG. 2B), a balloon may be inserted into the side branch through the mesh of the stent and the balloon inflation creates an opening on the stent's mesh at the level of the orifice of the side branch. While the flow into the side branch is liberated, there are several disadvantages using this method. First, it requires additional manipulations and balloon inflations. Since the stent has been already deployed with a series of balloon inflations (pre-deployment, during deployment, post-deployment), the additional balloon inflations just add further injuriousness to the whole procedure. Secondly, the additional manipulations may dislodge the stent from its position, and /or some of the struts of the stent will protrude into the lumen of the main branch stent and causes turbulence and flow obstruction.

As another example, as illustrated in the T stenting embodiment (FIG. 2C), after a straight stent 2 is placed into the main branch another straight stent 1 is placed into the side branch. In this case the main branch stent is not deformed to create an opening to the side branch, therefore the stent struts cause partial flow obstruction to the side branch. This solution is indicated when the side branch also has a stenosis that should be treated. However, another disadvantage of this method is that gap 220 is created in the proximal part of the side branch with no stent coverage. Therefore, any kind of existing ostial lesions are not addressed or prevented from occurring in the long run. As another example, the “reverse T stenting” embodiment (FIG. 2D) is a combination of the previous two methods: it comprises first creating an opening on the mesh of the main branch stent 1 by balloon inflation then placing an appropriately sized straight stent 2 into the side branch. Because this is a combined method, the potential disadvantages stem from each of the components. Again, gap 220 provides no stent coverage at the proximal portion of the side branch.

As another example, another possible method available to address diseased bifurcations is to use the “Culotte” method (FIG. 2E). In this technique, a straight stent 1 is deployed into the main branch then another stent 2 is released through the mesh of the main branch stent into the side branch. While the method can provide free flow to the side branch, and also can treat any diseased portion of the side branch vessel, the main branch stent should be deformed and also two layers of overlapping stents will be present in the proximal part of the main branch making this segment very rigid. The overlapping wire struts can impose problems with proper inner layer forming (endothelization) on the stents. The side branch stent's wires will cause partial flow obstruction in the main branch. Although theoretically with another balloon the struts that cause this partial flow obstruction can be pushed away (creating an opening on the side branch stent toward the distal portion of the main branch), the required manipulations can result in more injuriousness, further deformation of the main branch stent that in turn can cause protruding wire struts, partially obstructed flow, turbulence, in-stent restenosis, and embolism.

As another example, the “Y stenting” method (FIG. 2F) uses three separate stents to cover the bifurcated anatomy and creating gaps in between the adjacent stents while the “V stenting” method utilizes only two stents which are deployed into the distal portion of the main branch and the side branch, respectively. As another example, the “Crush” stenting method (FIG. 2G) uses two stents: one stent 2 is deployed into the main branch and through it another stent 1 is deployed into the side branch. While this solution eliminates the double layers in the proximal portion of the stent assembly, additional balloon inflation should be used to crush the proximal portion of the side branch stent into the main branch stent to cover the orifice of the side branch. Among other disadvantages, this method results in stent deformation (main branch), uneven arrangement of the struts (side branch stent), and additional injuriousness to the vessels and changes in flow patterns.

As still another example, the “kissing” stent method (FIG. 2H) comprises two simultaneous stent placements where the proximal portions of these stents are at the same level; one stent is deployed into the main branch and the second stent into the side branch. This technique requires simultaneous balloon inflations during the stent placements. If the stents were deployed separately, it would be very difficult or even impossible to maintain the original diameters and round cross-sections of the branches. An already deployed stent with its nominal diameter will hinder the placement of the second stent. The presence of the two stents eventually creates a bulging at the affected levels of the bifurcation that also add to the damage that the repeat previous balloon inflations have already caused. At the sides of the stents facing toward each other the flow pattern can change resulting turbulence with the known possible consequences. In addition, the cross-sections of the stent deployed parallel to each other will be rather elliptical and not round in shape. This fact may also result in changes in flow pattern.

As is known in the art, a stent may generally be characterized as being a balloon-expendable stent or a self-expanding stent, depending upon how deployment is affected. Self-expanding stents and balloon-expanding stents differ in many respects and have very different mechanical and dynamic properties. Various technical papers have discussed these differences in detail, and are generally known to those of skill in the art.

Balloon expanding stents are manufactured in the crimped state and are expanded to the vessel's diameter by inflating a balloon, thus plastically deforming the stent. In contrast, self-expanding stents are manufactured at the vessel diameter (or slightly above) and are crimped and constrained to the smaller diameter until the intended delivery site is reached, where the constraint is removed, and the stent deployed. Accordingly, balloon expanding stents resist the balloon expansion process, whereas self-expanding stents assist vessel expansion. In other words, while self-expanding stents typically become part of the anatomy and act in harmony with native vessels, balloon-expanding stents change the geometry and properties of the anatomy. From the Applicant's perspective, self-expanding stents assist, while balloon-expanding stents dictate.

While balloon-expandable stents generally reach their maximum diameter at the time of deployment (depending on the pressure of inflation), self-expanding stents typically continue to expand post deployment, reaching their maximum diameter several days to weeks later post deployment. With balloon-expandable stents, the vessel should be overdilated (e.g., +10-20%) to overcome the artery's “elastic recoil” (e.g., the tendency for the artery after dilation to return back to its original diameter). The overdilation is necessary to achieve a good apposition of the balloon-expandable stent. Without subsequent balloon dilatation, a balloon-expanding stent may become smaller in diameter over time (chronic recoil). Furthermore, the lesion should be pre-dilated before placement of the balloon-expanding stent because these stents inherently lack any expansile force. In addition, the placement of these stents frequently requires post-placement dilation to achieve optimal apposition of the stent to the vessel wall. Sometimes all of these steps (e.g., pre, during, and post deployment) consist of multiple balloon inflations. These multiple overdilations of the vessel is evidently much more injurious than the damage a self-expanding stent can cause during placement. A properly over-sized self-expanding stent, however, continues to apply a force acting to expand the vessel. Further, a self-expanding stent typically undergoes a negative chronic recoil (that is, a luminal gain), which means that a self-expanding stent continues to open over time, often remodeling the vessel profile. In general, self-expanding stents generally reside near and scaffold the outside of the vessel wall, while balloon-expanding stents remain near the lumen. The negative recoil of self-expanding stents may become an important advantage over drug eluting balloon expanding stents, in which the lumen may actually increase shortly after the deployment by inhibiting the neointima formation (new inner layer of the vessel), but this cell inhibition may result in a patchy covering layer over the stent leaving the balloon-expanding stent exposed to the flow of blood and resulting in in-stent restenosis in the long run.

Radial strength describes the external pressure that a stent is able to withstand without incurring clinically significant damage to the vessel lumen. Balloon-expanding stents can collapse if a critical external pressure is exceeded, potentially having serious clinical implications (stent crush resulting in obstruction of the lumen). On the other hand, self-expanding stents generally have no strength limitation and elastically recover even after complete flattening or radial crushing. Thus, self-expanding stents are ideally suited to superficial locations, such as the carotid and femoral arteries.

Stiffness is defined as how much the diameter of a stent is reduced by the application of external pressure. Axial stiffness is directly reflected in bending compliance. A balloon-expanding stent is typically stiffer than a self-expanding stent of identical design because of the lower elastic modulus of nitinol. Self-expanding stents are much more compliant than balloon-expanding stents of identical design, which is applicable for both delivery and deployment. Self-expanding stents typically adapt their shape to that of the vessel rather than force the vessel to the shape of the stent. Forcing a vessel into an unnatural shape, even if straight and aesthetically pleasing, can lead to high contact forces at the ends of the stent. Flexible links that are plastically deformed during bending accumulate damage and can fracture quickly as a result of fatigue.

Acute recoil refers to the reduction in diameter immediately observed upon deflation of a balloon. A balloon-expanding stent recoils after balloon deflation, whereas self-expanding stents assist balloon inflation (if needed post deployment) and thus there is no recoil of a self-expanding stent. As applied to a vessel, however, both devices generally recoil due to the springback forces of the vessel and constrictive forces of a significant stenosis.

The delivery profile of a balloon-expanding stent is typically dictated by the profile of the balloon upon which it is mounted. In contrast, self-expanding stent profiles are typically dictated by the strut dimensions (e.g., the width) required to achieve the desired mechanical performance. Current minimum profiles of the two types are very similar, but self-expanding stents have the greater potential to reduce in size. This is expected to play an important role in neurovascular stenting, where both delivery profile and flexibility are essential.

While bending, crushing, and stretching fatigue considerations is often ignored for stents, these factors can be very important in certain indications. One extreme case is the femoro-popliteal artery, but such issues can also be important in coronary vessels because of the systolic expansion of the heart (thereby stretching the stent). For example, older generations of coronary stents were very rigid in the axial direction and not subject to axial fatigue; newer more flexible generations, however, can experience axial deformations and may be prone to fatigue damage. Nitinol performs far better than any other known metal in displacement-controlled environments such as these, which ultimately may mean that this more fatigue-resistant metal offers further advantages under various circumstances.

Newer stent platforms (both balloon and self-expanding) have been designed to increase flexibility, radial strength, torsion, and lengthening or shortening of the vessel, with decreased rates of stent fracture and restenosis. For example, newer-generation self-expanding nitinol stents have a superelastic metallic alloy of nickel and titanium and thinner struts, thereby resulting in better deliverability and less in-stent restenosis.

As is known in the art, the following stents are generally known to be a balloon-expandable stent: Antares (by TriReme Medical), Twin-Rall (by Invatec), Multi-Link Frontier (by Abbott), Nike Croco and Nile Pax (by Minvasys), Petal (by Boston Scientific), SideKick (by Y-Med), SLK-View (by Advanced Stent Tech), and Tryton (by Tryton Medical). As another example, Cordis offers a Flash Ostial balloon system that uses a special balloon catheter with two different diameter balloons. A distal balloon is used to deploy the balloon expandable stent and a proximal balloon is used to create a flange at the level of the orifice. During installation, one or more markers may be used to place the stent at the right positions relative to the ostium. For example, three marker bands may be used to ensure that the middle marker is at the ostium, a distal marker is proximal to the distal edge of the stent, and a proximal marker is outside of the guide catheter. As another example, Ostial Pro is a stent positioning system offered by Merit Medical for an aorto-ostial lesion (among others). The Ostial Pro stent comprises a stent that is expanded/flared by an inflatable balloon inserted into the stent. A plurality of expanding legs helps set the plane of the ostium for setting/positioning the stent. As another example, the Tryton Side Branch Stent is deployed in the side branch artery using a standard single-wire, balloon-expandable stent delivery system. The stent may comprise a main branch portion and a side branch portion with a transition portion between the main branch and side branch. Each of the portions may be separately inflated. For deployment, a separate wire is inserted into the main branch as well as the side branch and the side branch stent is positioned. The main branch is then re-wired and a drug eluting stent (DES) is deployed, and the stent is expanded with a kissing balloon. Thus, the Tryton stenting system may use a first stent placed in the side branch followed by a second stent placed in the main branch (which may be deployed through the first stent), each of which may be inflated with a kissing balloon. As another example, Boston Scientific offers a bifurcation stent system by the name of Taxus. The Taxus system is advanced over a guidewire through the coronary vasculature to deliver and dilate the stent at the target lesion location. Following stent deployment, the delivery balloon may be inflated with additional pressure in order to optimize the stent luminal diameter and strut apposition. Boston Scientific also offers a Taxus petal Stent, which is similar to the Taxus Stent. Still further, there are other dedicated bifurcation systems, including a stent with dedicated side branches (such as those offered by Tryton and Capella), a stent with fixed angles (such as those offered by Medtronic and Biosensors), and a stent with a side hole (such as those offered by Boston Scientific, Abbott, Stentys, TriReme, Minvasys, and others).

Percutaneous transluminal coronary angioplasty with stenting has now become one of the cornerstones of treatment for coronary artery disease (CAD). Self-expanding stents and balloon-expandable stents are routinely used in peripheral arterial disease, although balloon-expandable stents have become the stent of choice for coronary arteries. The use of third-generation drug-eluting stents (DES) is currently the preferred method of treatment for all patients with coronary artery disease.

In general, most conventional techniques for treating bifurcation lesions use a balloon expandable stent. This is no surprise, as there is an existing bias in the coronary stent market that favors third generation DES balloon expanding stents over currently available self-expanding versions. For example, one reason that self-expanding stents are not favored is that most of these self-expanding stents are nitinol slotted tube designs that are plagued by undesirable features such as having a weak expansile force, are crash-prone, easily break, and are not flexible enough, etc. As another example, balloon-expanding stents have traditionally provided more optimal placement within the vessel, and traditional balloon-expanding stents have provided less recoil than self-expanding stents when placed in particular locations.

However, the deployment of the balloon expanding stents are overly injurious and requires a series of overdilation of the vessel. Further, while the DES coating used with these balloon expanding systems aims to control the reactive abundant neointima formation by inhibiting the growth of the endothelial cells (which may work for a short time), the patchy and partial endothelization may result in frequent in-stent stenosis and thrombosis in the long run. For example, the stent wires may not be completely covered, and the bare wires may be a source of thrombus formation (e.g., in-stent restenosis). Another negative aspect of using DES is that while the Everolimus inhibits growth factor-stimulated cell proliferation leading to inhibition of cell metabolism, growth, and proliferation by arresting the cell cycle at the late G1 stage, it can sensitize patients and also the use of such drug-treated stent is contraindicated in patients with any immune compromise. While the amount of the DES drug, which is released from the stent's polymer coating, is said to have clinically insignificant systemic dose, it has the potential to cause some deleterious effect in some individuals. Further, while the balloon expandable stents in general have greater outward forces after placement than a typical self-expanding stent, their ability to withstand external forces are very limited and they suffer with crush-prone designs. For example, the slotted tube nitinol balloon expanding stents are inherently brittle and breakable and are not resistant to fatigue. Further, how these balloon expandable stents are designed (such as to be flexible) further increase the possibility of fatigue (by thin connecting struts as bridges).

The following stents are generally known to be a self-expanding stent: Axxess (by Devax), Sideguard (by Cappella), Sparrow (by CardioMind), and Stentys (by Stentys). Others include the Wallstent, which was the first self-expanding stent used in humans and also the first stent used in the coronary arteries. The Wallstent is made of a cobalt alloy with an inner platinum core, and has since been replaced by the Magic Wallstent. Other self-expanding stents include the Symbiot stent, the Protect stent, and the Igaki-Tamai stent. Each of these well-known self-expanding stent devices—and their use and application—are incorporated herein by reference.

Several self-expanding devices for bifurcation lesions are similarly described in U.S. Pat. No. 7,018,401 (“the '401 Patent”) and U.S. Pat. No. 8,739,382 (“the '382 Patent”), each incorporated herein by reference. Further, each of the '401 and '382 Patents provides a summary of some of the prior art relevant to the present disclosure, the prior art which is incorporated herein by reference.

The '401 Patent is directed to a self-expandable, woven intravascular device for use as stents, filters, and occluders for insertion and implantation into a variety of anatomical structures. The devices may be formed from shape memory metals such as nitinol, may be formed from a single wire, and may be formed by either hand or machine weaving. The devices may be created by bending shape memory wires around tabs projecting from a template and weaving the ends of the wires to create the body of the device such that the wires cross each other to form a plurality of angles, at least one of the angles being obtuse. In general, the proximal end of the straight stent is created by back-weaving the wire strands and then welding them at the appropriate points. As one example, FIGS. 1A, 1B, and 1C of the '401 Patent illustrate examples of a prior art stent, which are reproduced as FIGS. 3A, 3B, and 3C in the present disclosure, respectively. As reproduced from the '401 Patent at col. 15, lines 20-34 (and incorporated herein by reference):
“Body 10 is both radially and axially expandable. Body 10 includes front or distal end 12 and rear or proximal end 2. As shown in FIG. [3]A, end 12 has a plurality of closed structures. These closed structures may be small closed loops 6 or bends 8 (FIG. [3]B). Both bends 8 and small closed loops 6 may be formed by bending a wire 5 at a selected point located between the ends 7 of wire 5 (FIG. [3] C shows small closed loops 6). For most applications, the selected point of the bend or small closed loop may be close to the midpoint of wire 5, as shown in FIG. [3] C with respect to small closed loop 6. FIG. [3]C also shows both ends of wire 5 being located proximate end 2 of body 10 (although the remainder of body 10 is not shown). Body 10 is formed by plain weaving wires 5 . . . ”

The '382 Patent is directed to a woven, self-expanding stent device that has one or more strands and is configured for insertion into an anatomical structure. The device includes a coupling structure (that is not a strand of the device) that secures two different strand end portions that are substantially aligned with each other. FIGS. 1, 2, and 3 of the '382 Patent illustrate one embodiment of making of a prior art stent, which is incorporated herein by reference, that discloses weaving a stent from six strands (wires) that possess twelve strand halves 10. There are no free strand ends at the end of the stent device. After heat treatment, the device can be immediately quenched in deionized water until cool. FIG. 3 of the '382 Patent shows device 100 after half of the twelve loose strand ends have been backbraided. The coupling structures that are used (for stents, the number of coupling structures will preferably equal the number of strands) may be axially aligned with the strands, such as those coupling structures displayed in FIGS. 3, 4A, 4B, 6, and 7 of the '382 Patent, incorporated herein by reference.

Abbott offers a stenting system known as Supera. The instructions for use of the Supera Stent is publicly available and known to those of skill in the art and is incorporated herein by reference. The Supera Peripheral Stent System consists of a closed end, braided self-expanding stent made of Nitinol (nickel-titanium alloy) wire material that is pre-mounted on a 6Fr delivery system. The stent typically does not include radiopaque markers, but to increase the radiopacity of the stent, the wire strands can be nitinol microtubings with a platinum core.

The Supera stent is a platform stent. In other words, the Supera structure allows the design of any size and/or length stent that is needed for the different vascular and non-vascular regions of the body, including the coronary artery. In general, any structure can be made from the appropriate number and size of wires to create a mesh that fits best given a particular anatomy's needs. For example, for coronary application, a Supera stent can be produced with a smaller number of wires than traditionally used; instead of twelve wires (six pairs), the stent may use six, eight, or ten wires to form the mesh. The mesh tightness can be adjusted according to the specific need, from open cell design to very tight mesh where the wire strands obtuse angles approaching 180 degrees. If a stent is made with a mesh size that is looser than the original design (that is the obtuse angles in the mesh are reduced), that will be the stent nominal diameter that is imprinted to the wires by the heat treatment. Such a stent may still feature a significant radial force what it is deployed with in nominal diameter.

The Supera stent sizes are labeled based on the outer stent diameter. A stent should initially be chosen such that its labeled diameter matches the reference vessel diameter (RVD) proximal and distal to the lesion. Final stent selection should be confirmed after lesion pre-dilation: if possible, the stent diameter should match the prepared lesion diameter 1:1. Due to the mechanical behavior of the woven Supera stent, the stent should not be oversized by more than 1 mm relative to the RVD. This ensures optimum deployment of the Supera stent, maximizing radial strength, and assisting in accurate stent length deployment. Choosing a labeled diameter to match the reference vessel diameter, then appropriately preparing the vessel to match that stent's diameter will result in a stent that is properly sized to the vessel. The vessel should be prepared utilizing standard angioplasty technique using a balloon size greater than or equal to the stent diameter. The post-dilated vessel should be at least the size of the stent diameter.

The Supera stent mimics the natural structure and movement of the anatomy. The innovative, interwoven nitinol design creates a stent that supports rather than resists the vessel. By pre-dilating and matching the stent and vessel 1:1, the Supera stent supports the vessel with minimal chronic outward force. The Supera stent has increased strength and flexibility, with more than four times the compression resistance of typical standard nitinol stents (e.g., slotted tube designs). In severely calcified lesions, the Supera stent has visible compression resistance, maintaining a round, open lumen for normal, healthy blood flow in challenging anatomy.

The over-the-wire stent delivery system for Supera is compatible with a 0.014″ and a 0.018″ guide wire and comes in lengths of 80 cm and 120 cm (6Fr). The delivery system includes a reciprocating mechanism (e.g., stent driver) that incrementally moves the stent distally out of the outer sheath. This motion allows for the distal end of the stent to first come in contact with the targeted vessel, setting the distal reference point, and continues to feed the stent out of the sheath as the target wall is exposed by the proximal movement of the catheter. This stent deployment is achieved by the reciprocation of the thumb slide located on the handle. On the final stroke, the deployment lock is toggled, and the last deployment stroke is made.

A Supera based coronary stent has a strong expansile (outward) force that can be easily adjusted by carefully selecting the size and the number of the wires for the mesh. The stent structure allows to create stents between a relative open cell arrangement and a very tight mesh size. The stent is biomimetic; that is, the stent can easily follow and accommodate the most tortuous anatomy while its inner diameter is never compromised. The Supera stent has perfect conformability. The Supera stent can withstand repeated pulsatile outer compressing forces, as well as axial movements (shortening/elongation) which is a factor in the coronary arteries on the surface of the ever-pulsating heart. The Supera stent is known to be able to endure very strong complex forces in the most challenging anatomy, namely in the femoropopliteal region. It can withstand compressive forces (popliteal artery), torque, shortening and elongation (femoral artery) even when these forces are present in combination. The stent may allow to eliminate the need for pre- and also post placement dilation, therefore its placement is much-much less injurious that that of the balloon expandable stent.

The Supera stent offers numerous benefits over other self-expanding stents and in particular balloon-expanding stents. In particular, Supera based coronary stents can overcome many of the inherent problems of balloon-expandable stents, such as high-pressure balloon inflations, overexpansion of a narrow segment distal to the stent, increasing the risk of an edge dissection, and underexpansion of a proximal segment resulting in poor stent apposition. Further, Supera based coronary stents eliminate the need for multiple very injurious balloon dilations and makes unnecessary the use of a DES coating that (while it has been proven to be advantageous to restrain neointima formation and increase lumen gain in the early phase of post-stenting) may result in patchy coverage of wire struts by neointima that can negatively influence in-stent restenosis in the long run. Self-expanding stents in general, and the woven nitinol stent in particular, embed deeply in the vessel wall reaching the muscular layer. As a result, the neointima coverage can be complete. That is important to avoid in-stent restenosis later. Even a larger layer of the neointima will be adequately compensated by the fact that the stent making lumen gain after deployment for days or weeks. Still further, because balloon expandable stents cannot be used in vessels with a diameter larger than 4.5 mm, these lesions cannot be treated with them. The Supera based coronary stent's versatility in diameter and length allows treatment over a wide range of lesion sizes and configurations.

Conventional stents—whether balloon or self-expanding—possess certain shortcomings that inhibit their ease and range of use. In particular, the dominant use of a balloon expandable stent in the coronary field is problematic, particularly in view that some self-expanding stents have several advantageous features over the balloon expanding counterpart. Self-expanding stents in general, and in particular a Supera based woven nitinol stent, provides several characteristics that make these stents more advantageous for the coronary application than the balloon expandable stents.

Despite the apparent benefits of a Supera based stenting system, it fails to address ostial lesions and lesions of branching and bifurcations. A straight stent cannot solve the problems that these anatomical situations pose.

Stents are necessary for both the arterial system and the venous system. Relative to the arterial system, the venous system is characterized by low pressure, low velocity, large volume, and low resistance. The heart, pressure gradients, the peripheral venous pump, and competent valves interact together to overcome the hydrostatic pressure induced by gravity. The larger veins serve as the primary capacitance vessels where most of the blood volume is found and where regional blood volume is regulated. Venous stenosis is intimal hyperplasia and fibrosis causing progressive vessel narrowing and outflow obstruction. Venous stenosis most commonly affects the axillary, brachial, cephalic, or brachiocephalic veins of the upper extremities, or the superior vena cava, but can also affect the central veins in the abdomen and the pulmonary artery and veins. Common causes are placement of central venous catheters, pacemaker leads, hemodialysis catheters, prior radiation, trauma, or extrinsic compression. The use of venous stents is a medical necessity in patients with disabling or life-threatening occlusive or stenotic disease of the central veins that extend from the iliofemoral veins to the subclavian veins.

There are numerus prior art stenting applications for venous stenting. The most extensive clinical experience within venous stenting has been off-label use of the Wallstent (available from Boston Scientific). The Wallstent is available in large diameters and has an Elgiloy (similar to stainless steel) braided construction that provides flexibility. Unfortunately, because of the flexibility of the Wallstent, when constricted, its length varies resulting in decreased deployment accuracy. Accurate deployment is also limited by foreshortening of up to 40%. If there is compression near the end of the stent, as is often the case near the ilio-caval junction, the Wallstent may form a narrowed cone shape, which decreases flow, or the stent can migrate as it is squeezed away by a compressive lesion. Another pitfall of the Wallstent is in the setting of bilateral iliac stenting. In order to decrease problems with jailing of the contralateral side or narrowing of bilateral stents in the inferior vena cava, multiple techniques have been developed including the double barrel, fenestration, and Z stent techniques. Unfortunately, none of these techniques are ideal and may result in the need for reintervention.

The prior art also includes dedicated venous stents, many of which comprise self-expanding nitinol stents. For example, the Cook Zilver Vena venous stent has an open cell design and is available in 14-16 mm diameters and 60-140 mm lengths. The stent has a 7-French platform that is compatible with 0.035″ wires. The open cell design affords flexibility and the stent has minimal foreshortening. The Veniti Vici Venous stent, distributed by Boston Scientific, has a closed cell design with uniform end-to-end shape and strength. It is available in 12-16 mm diameters and 60-120 mm lengths. It has a 9-French platform compatible with 0.035″ wires. The closed cell design and sinusoidal strut rings give strength while the alternating curved bridges afford flexibility. It is a strong stent, with a sufficient surface area, performing well in May-Thurner syndrome patients, with good stent integrity. The Optimed sinus-Venous stent has a hybrid design trying to balance the need for both radial force and flexibility. The stent comes in 10-18 mm diameters and 60-150 lengths with a 10-French platform. With the sinus-Venous device, one has to be very accurate with the initial deployment because there is no change of repositioning. It is a flexible stent and with the correct deployment technique it also has a high crush resistance. The sinus-XL Flex stent is easier to deploy, though it is less flexible, and it tends to kink at the flex points. The Optimed sinus-XL stent has a closed design affording high radial force. The stent comes in 16-36 mm diameters and 30-100 mm lengths with a 10-French platform. The sinus-XL in intended for large linear vessels including the aorta and the inferior vena cava. An Optimed sinus-XL 6F also has been developed with diameters of 14 and 16 mm. Another stent, the Optimend sinus-XL Flex comes in large diameters (14-24 mm) but has an open cell design to afford it more flexibility. It comes in 40-160 mm lengths and also has a 10-French platform. The Optimed sinus-Obliquus has a hybrid design with a closed cell design oblique-shaped central end, an open cell design mid-segment, and an anchor ring at the peripheral end. The closed cell oblique segment allows for increased radial force and crush resistance at the iliocaval stress point while minimizing overlap of the contralateral common iliac vein. The open cell design of the mid-segment provides flexibility and conformity to the stent. The peripheral end anchor helps with stent fixation. The sinus-Obliquus stent, with its 10-Fr platform, comes in 14- and 16-mm diameters and 80-150 mm lengths. Medtronic has more recently initiated a dedicated venous stent investigational device exemption study, the ABRE IDE study. The Abre venous self-expanding stent has an open cell design with 3 points of connection between cells to afford it flexibility and conformity. This stent system has a triaxial shaft design to aide with delivery. This device is delivered through a 9-Fr system and will be available in diameters up to 20 mm.

Despite the recent advance in the venous stenting field still no ideal venous stent exists. The majority of the recently developed venous stents designs lacks several important features of a desirable venous stent. For example, most of the conventional venous stents are nitinol slotted tube designs that inherently lack adequate radial forces, equipped with connective struts to facilitate flexibility but these elements simultaneously make the structure vulnerable to outer forces, bringing in factors of long-term fatigue etc. There is a real need to develop stents that feature the most requirements for an ideal venous stent.

The statements in this section are intended to provide background information related to the invention disclosed and claimed herein. Such information may or may not constitute prior art. It will be appreciated from the foregoing, however, that there remains a need for an improved method, device, and system for treating ostial lesions and branching anatomies, particularly with self-expanding stents. A need exists for an improved method, device, and system for treating complex lesions including branching and bifurcated anatomies in the arterial system as well as the venous system. A need exists for a self-expanding stent and stent system that addresses problems such as high-level injuries of the balloon expandable stents, branching/bifurcated lesions, ostial lesions, and lesions with complex anatomy. Such disadvantages and others inherent in the prior art are addressed by various aspects and embodiments of the subject invention.

### SUMMARY OF THE INVENTION

This disclosure provides a method, system, and apparatus for self-expandable, woven intravascular devices for use as stents (both straight and tapered) in a variety of anatomical structures, including vessels in the arterial and/or venous system. The stent devices may be formed from shape memory metals such as nitinol. The disclosed flange stent may be used as an ostial stent that provides an accurate and optimal stent placement into a side branch of a branching anatomy. The disclosed flange stent may also be part of a modular stent assembly and/or utilized with other stents that are not flanged. The disclosed stent device may also be used for main branch and side branch applications. A first stent may be placed in a first vessel (such as a main branch), and a second stent (which may be a flanged stent) may be placed in a second vessel (such as a side branch) through a wall of the first stent. The devices may be formed by either hand or machine weaving, and may be created by bending shape memory wires around a template with a flanged portion, heat treating a substantially straight stent around a template with a flange, or altering the pick-count (weave density) over a straight template (mandrel) followed by heat treating to achieve the desired flanged end.

Disclosed is a stent for treating ostial lesions, comprising a body with a first end and a second end and a flange at either the first end or the second end, wherein the flange is approximately perpendicular to the axis of the body. The stent may be self-expanding and may comprise a plurality of shape memory wires woven together. The stent may be configured to be deployed in an artery or vein.

In one embodiment, a diameter of the flange is greater than a diameter of the body. The flange may comprise a rim or may comprise a rounded flange. In some embodiments, the stent may comprise a curved transition portion between the flange and the body. In one embodiment, a body of the flange stent has a tubular shape, a substantially uniform diameter, and/or a substantially tapered shape. A body portion and a flange portion of the stent may be contiguous and may be made of the same or different materials. The flange portion may have a together mesh than a body portion of the flange stent, while in other embodiments the flange may comprise a looser mesh than the body.

Disclosed is a modular stent system, comprising a first stent that is substantially straight or tapered and a second stent coupled to the first stent. The second stent may comprise a body with a first end and a second end, and a flange at either the first end or the second end, wherein the flange is approximately perpendicular to the axis of the body. In one embodiment, the first stent and the second stent are configured to be coupled together inside the anatomical body. In one embodiment, the first stent is configured to be deployed prior to the second stent. In one embodiment the second stent is configured to be deployed through a mesh of the first stent. In one embodiment the first stent is configured to be positioned in a first vessel and the second stent is configured to be positioned in a second vessel, wherein the second vessel is a side branch of the first vessel. The first vessel may be a parent artery or parent vein. In one embodiment the second stent is configured to maintain the patency of the side branch. In one embodiment, the first vessel and the second vessel form a bifurcation, wherein a diameter of the second vessel is less than a diameter of the first vessel.

In one embodiment, multiple flange stents may be coupled to a primary stent, which itself may or may not be flanged stent. In one embodiment, the stenting system may comprise a first stent (which may be a first flanged stent or a substantially straight body stent), a second flange stent coupled to the first stent, and a third (or more) flange stent coupled to the first stent. The first stent may be positioned in a first vessel (which is a parent vessel), while the second stent and third stent may be positioned in a second vessel and third vessel, each of which are branching vessels to the first vessel. Each of the second and third stents may be deployed through a side wall or mesh of the first stent.

Disclosed is a method for deploying a flange stent, comprising inserting a first stent into a first vessel, deploying a second stent through a side wall of the first stent, and positioning the second stent proximate to a branching portion of the first vessel. The first stent may comprise a body portion that is substantially straight or tapered and the second stent may comprise a flange end and a flange body. In one embodiment, the method may comprise expanding the flange stent to contact walls of the branching portion of the first vessel. In one embodiment, the method may comprise inserting the flanged end of the flange stent through the first stent before the body of the second stent. In one embodiment, the method may comprise inserting the body of the flange stent through the first stent before the flanged end. In one embodiment, the method may comprise deploying a second flange stent through a side wall of the first stent into a second vessel, wherein the second flange stent comprises a body coupled to a flanged end and positioning the second flange stent proximate to a branching portion of the second vessel.

Disclosed is a method for deploying a flange stent, comprising inserting a guide wire into a first vessel and inserting a catheter with a flange stent over the guide wire, positioning the flange stent proximate to a side branch of the first vessel, and expanding the flange stent to contact the first vessel walls. The flange stent may comprise a body portion that is substantially straight and a flange portion on one end of the body portion. In one embodiment, the method may comprise expanding the flange stent to contact walls of the side branch. In one embodiment, the method may comprise inserting the body portion of the flange stent before the flange portion of the flange stent. In one embodiment, the method may comprise inserting the flange portion of the flange stent before the body portion of the flange stent. In one embodiment, the method may comprise deploying the flange stent by identification of one or more radiopaque markers attached proximate to the flange portion. In one embodiment, the method may comprise inserting a first stent into the first vessel, wherein the first stent comprises a body portion that is substantially straight or tapered.

Disclosed is a method for rescuing a misdeployed flange stent, comprising advancing a balloon catheter over a guide wire to a first position within a vessel, positioning the balloon catheter in a first portion of a flange stent, inflating the balloon catheter to contact an inner surface of the flange stent, advancing the inflated balloon catheter distally over the guide wire to a second position within the vessel to cause movement of the first portion of the flange stent, and removing the balloon catheter and guide wire. In one embodiment, the first portion of the flange stent comprises a body portion of the flange stent. In one embodiment, the second position causes a flange portion of the flange stent to abut an orifice of a side branch of the vessel. In one embodiment, the method may comprise compressing a body portion of the flange stent, which may create a tighter mesh in a proximal portion of the flange stent than a distal end of the flange stent.

Disclosed is a method of forming a flange stent, comprising providing a stent template with a first section that is substantially straight and a second section that comprises a flange, weaving a plurality of strands around the first section and the second section of the template, and forming a stent that comprises a body portion and a flange portion with at least some of the plurality of strands. In one embodiment, the body portion and the flange portion are contiguous. In one embodiment, the body portion and the flange portion comprise the same plurality of strands. In one embodiment, the plurality of strands comprises one or more platinum cored microtubings. In one embodiment, the method may comprise inserting one or more radiopaque markers at the flange portion of the stent. In one embodiment, the method may comprise heat treating the body portion and the flange portion to program the final shape of the stent after the weaving step, which may include heat treating the body portion and the flange portion at approximately the same time for a predetermined heat treatment time. In one embodiment, the flange portion and body portion are comprised of the same plurality of strands. In one embodiment, the flange portion is approximately perpendicular to the axis of the body portion. In one embodiment, the flange portion is woven with a tighter mesh than the body portion, while in other embodiments the flange portion is woven with a looser mesh than the body portion.

Disclosed is a method of forming a flange stent, comprising forming a substantially straight stent by weaving a plurality of strands, providing a stent template with a first section that is substantially straight and a second section that comprises a flange, coupling the substantially straight stent to the stent template, and heat treating a portion of the substantially straight stent to form a flanged portion of the substantially straight stent.

Disclosed is a method of forming a flange stent, comprising forming a substantially straight stent by weaving a plurality of strands over a substantially straight template (mandrel) with a first end and a second end, increasing the pick-count (weave density) between a portion of the first end and second of the template, heat treating the substantially straight stent, and removing the substantially straight stent from the template (mandrel) to form a flanged portion of the substantially straight stent.

Disclosed is a method of deploying a flange stent with an expandable balloon, comprising inserting a guide wire into a first vessel, inserting a catheter with a flange stent over the guide wire, positioning a body portion of the flange stent within a side branch of the first vessel, expanding a balloon, and positioning a flange portion of the flange stent adjacent to an orifice of the side branch by expanding the balloon. The balloon may be located within the catheter. The flange stent may comprise a body portion that is substantially straight and a flange portion on one end of the body portion (such as a distal end).

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1D illustrate one schematic of a branching anatomic duct with and without prior art stents.

FIGS. 2A-2H illustrate a chart of various techniques to stent a bifurcation lesion.

FIGS. 3A, 3B, and 3C are reproduced from FIGS. 1A, 1B, and 1C, respectively, of U.S. Pat. No. 7,018,401.

FIGS. 4A-4D illustrate various schematics of a flange stent according to one embodiment of the present disclosure.

FIGS. 5A-5C illustrate schematics of a flange stent template according to one embodiment of the present disclosure.

FIGS. 6A-6D illustrate schematics of the phases of deployment of a flange stent in the left renal artery according to one embodiment of the present disclosure.

FIGS. 7A-7C illustrate schematics of a modular flange stent according to one embodiment of the present disclosure.

FIG. 8 illustrates a schematic of the aortic arch and three flange stents deployed in its large branches through the mesh of the aortic stent according to one embodiment of the present disclosure.

FIGS. 9A-9B illustrate schematics of an abdominal aorta and renal arteries where two ostial stents were deployed through the mesh of the abdominal aortic stent according to one embodiment of the present disclosure.

FIG. 10A illustrates various stents positioned within a typical Medina classification according to one embodiment of the present disclosure.

FIG. 10B illustrates various stents positioned within a Lefevre classification according to one embodiment of the present disclosure.

FIGS. 11A-11D illustrate schematics of a branching vessel where ostial stents are deployed in the side branches through the mesh of the stent that was previously deployed in the main branch according to one embodiment of the present disclosure.

FIGS. 12A-12D illustrate steps of a trailing end flange deployment of the disclosed flange stent device according to one embodiment of the present disclosure.

FIGS. 13A-13D illustrate steps of a leading end flange deployment of the disclosed flange stent device according to one embodiment of the present disclosure.

FIGS. 14A-14F illustrate one method for rescuing and correcting a misdeployed flange stent according to one embodiment of the present disclosure.

FIGS. 15A and 15B illustrate schematics of a delivery catheter with a stent according to one embodiment of the present disclosure.

FIGS. 16A and 16B illustrate schematics of a deployment method for a flange stent using a balloon according to one embodiment of the present disclosure.

FIG. 17 illustrates one view of the iliofemoral artery system with two modular flange stent assemblies installed therein, according to one embodiment of the present disclosure.

FIG. 18 illustrates one schematic of the disclosed modular stent assembly with multiple flange stents according to one embodiment of the present disclosure.

FIGS. 19A-19E illustrate various venous stenting applications for the disclosed flange stent according to one embodiment of the present disclosure.

### DETAILED DESCRIPTION

Various features and advantageous details are explained more fully with reference to the nonlimiting embodiments that are illustrated in the accompanying drawings and detailed in the following description. Descriptions of well-known starting materials, processing techniques, components, and equipment are omitted so as not to unnecessarily obscure the invention in detail. It should be understood, however, that the detailed description and the specific examples, while indicating embodiments of the invention, are given by way of illustration only, and not by way of limitation. Various substitutions, modifications, additions, and/or rearrangements within the spirit and/or scope of the underlying inventive concept will become apparent to those skilled in the art from this disclosure. The following detailed description does not limit the invention.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures, or characteristics may be combined in any suitable manner in one or more embodiments.

### SUMMARY

In one embodiment, disclosed is a flange stent that provides accurate and optimal stent placement into a side branch of a branching anatomy. The disclosed stent may be used all over the body, such as for ostial lesions or for bifurcating/branching anatomies, as well as for the arterial system and/or the venous system. Furthermore, the disclosed stent can be configured with different mesh tightness, radial force, diameter, and length, each of which may fit a wide variety of anatomical structures all over the body. In one embodiment, the variability in the mesh tightness, the number, and the size of the wires used to create the mesh offers limitless variations that serves the goal to produce the ideal stent for various applications.

The flange stent may be used alone or be part of a modular stent assembly in which one or more parts of the stent comprises or is coupled to a flanged stent. For example, for branching anatomies, a substantially straight stent (such as a Supera based stent) can be used with one or more of the disclosed flange stents. In one embodiment, a straight Supera (or other self-expanding substantially straight woven stent) based coronary stent is placed in a main branch first, then through the mesh of the main branch stent a flange sent is placed into a side branch. All commercially available stents (whether balloon expanding versions and/or self-expanding stents) are inferior to the disclosed solution that provides a flange stent with or without a Supera based coronary stent.

The disclosed flange stent provides numerous benefits. It offers a viable solution for stenting ostial lesions. It maintains the patency of side branches of an already stented vessel (parent artery or vein). It may be used to stent bifurcated lesions, that is, lesions that affect a vascular (or non-vascular) bifurcation.

The design and properties of self-expanding stents make them potentially suitable for interventions in various complex coronary lesions, including vein graft lesions, bifurcation lesions, tortuous vessels, left main disease, small vessels, and lesions causing ST elevation myocardial infarction (STEMI). In sum, the unique properties of self-expanding stents—and in particular the disclosed self-expanding flanged stent—make them an attractive and promising alternative to conventional techniques available for the treatment of coronary lesions.

In one embodiment, the disclosed flange stent is a self-expanding stent as opposed to balloon expandable stents typically used in the prior art. Self-expanding stents in general, and in particular a Supera-based woven nitinol stent, provides several characteristics that make these self-expanding stents more advantageous for the coronary application than prior art balloon expandable stents. Woven nitinol superelastic stents are ideal in respect to their physical characteristics. For example, they have robust redial forces that are even throughout the stent length, they have excellent conformability even in the most tortuous vasculature without compromising its lumen, they are crush-resistant, and they withstand tens of millions of pulsating forces without a break. In addition, the disclosed design allows for creating stents with a physical characteristic which is ideal for the given type of vasculature. As another example, deployment of the disclosed self-expanding stent is much less injurious than balloon expandable stents, and there is no need to use balloon inflations to get an appropriate apposition to the vessel wall. As another example, the self-expanding stent has much better conformability and is able to adapt to the given anatomical situation as compared to balloon stents (which generally straighten the stented segments because the balloon itself is not flexible). In particular, the mechanism of post dilation of the balloon expanding and the self-expanding stent is different—whereas the balloon expanding stent requires significant over-dilation of the vessel to achieve the appropriate apposition, the disclosed self-expanding stent inherently withstand any overdilation, that is, the inflation of the balloon only facilitate the stent to take on its maximum diameter. In one embodiment, if the disclosed stent size is determined precisely according to the diameter of the vessel (including any flanged portion), any possibility of over-dilation of the self-expanding stent is eliminated.

The disclosed self-expanding stent can overcome many of the inherent problems of balloon-expandable stents, such as high-pressure balloon inflations, overexpansion of a narrow segment distal to the stent, increased risk of an edge dissection, and underexpansion of a proximal segment resulting in poor stent apposition. Limitations of self-expanding stents include difficulty with precise placement, especially in ostial lesions, and inability to oversize the stent beyond its “set” diameter. These prior art stents, being bulkier, may also be difficult to deliver.

### Flange Stent

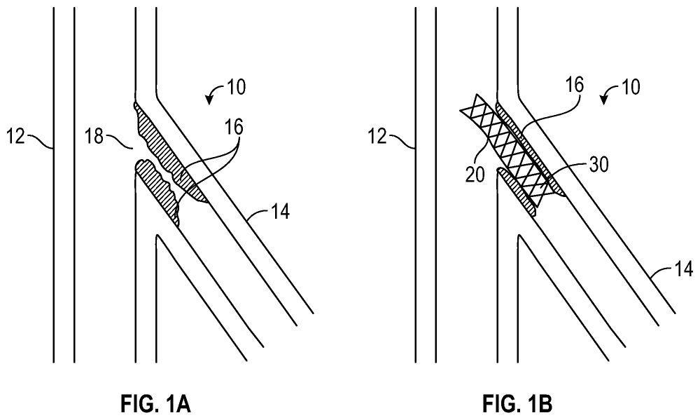

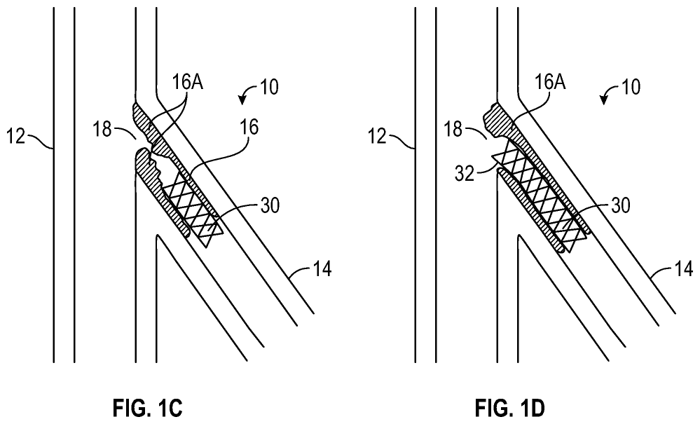

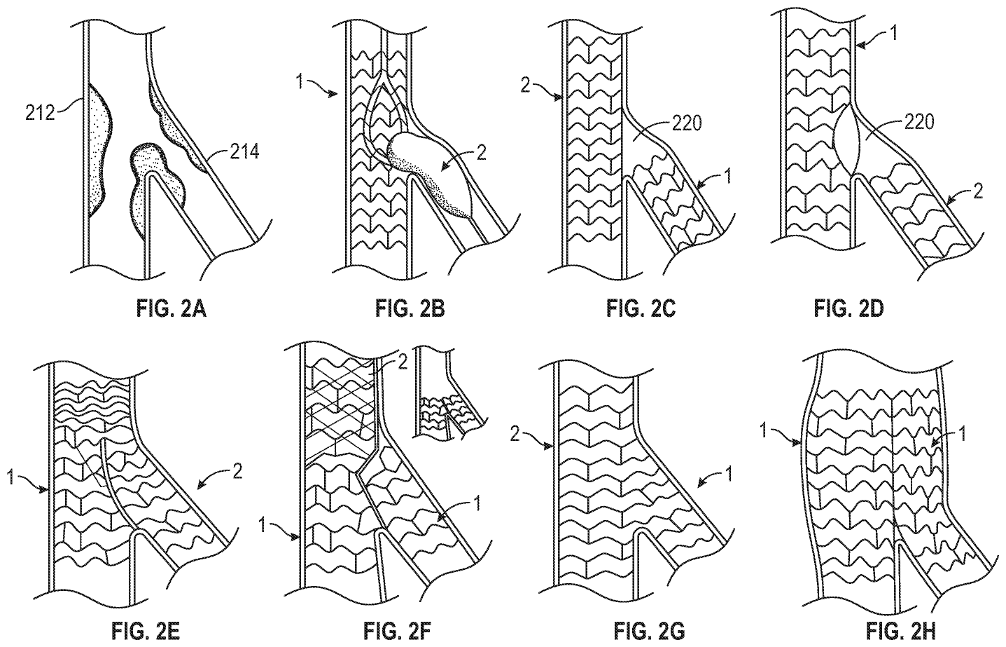

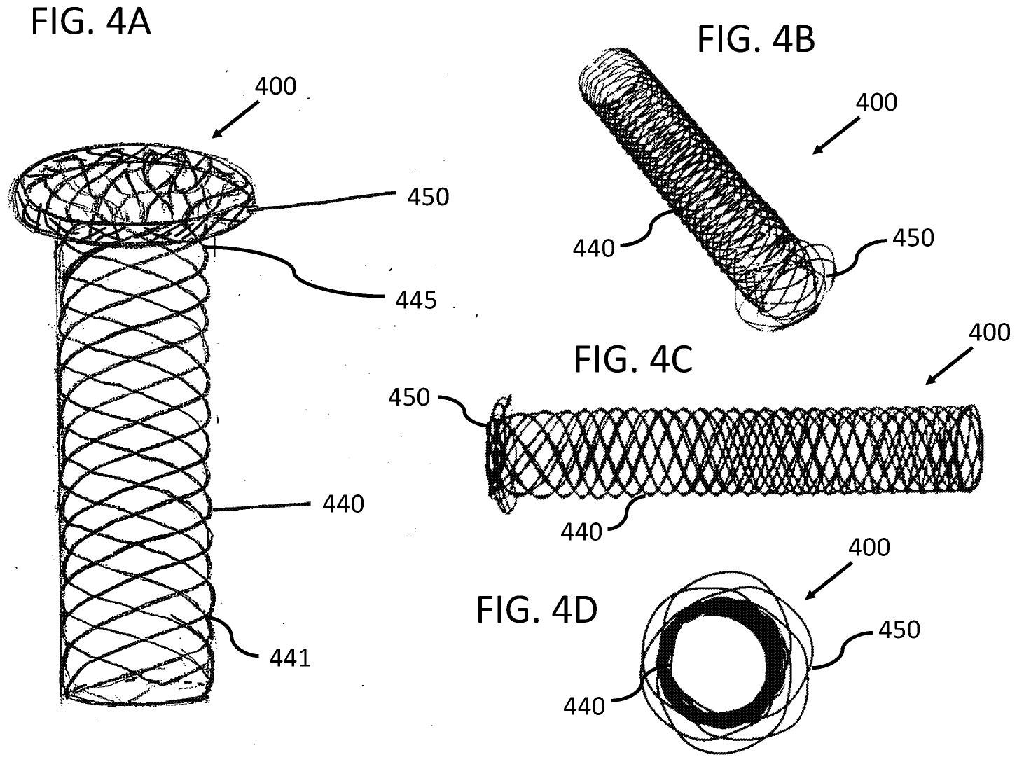

FIGS. 4A-4D illustrate various schematics of a flange stent according to one embodiment of the present disclosure. The disclosed flange stent may be used as an ostial stent that provides an accurate and optimal stent placement into a side branch of a branching anatomy and prevent any of the potential misplacements and issues described above in relation to FIGS. 1A-1D and FIGS. 2A-2H.

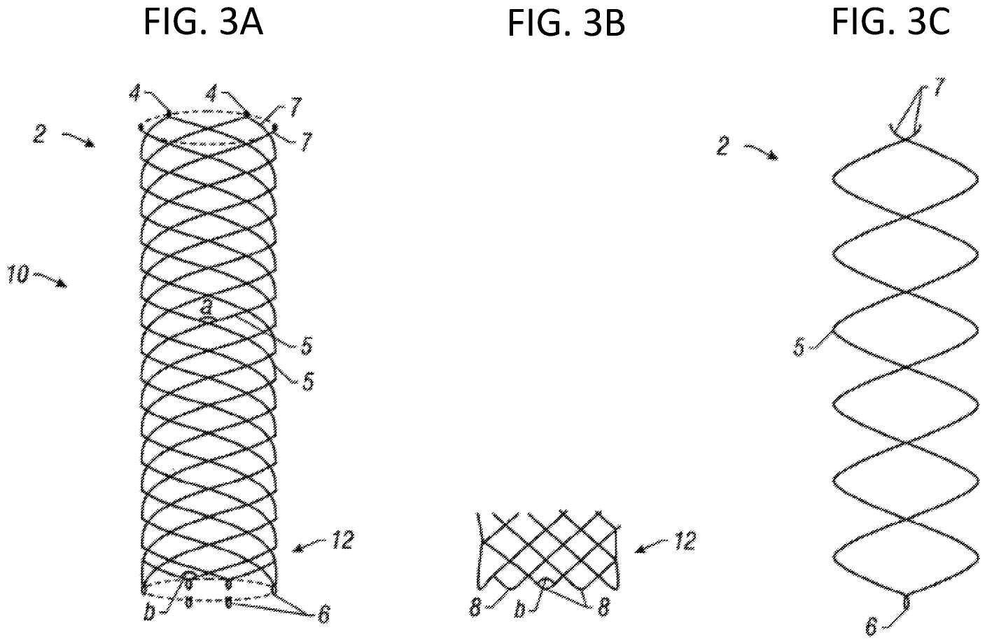

FIG. 4A illustrates one embodiment of flange stent 400 of the present disclosure. As disclosed herein, flange stent 400 is comprised of woven wires 411, which may be similar to that described in the '401 patent and the '382 patent, discussed above. See also FIGS. 3A-3C. The flange stent has a straight body 440 and flange portion 450 connected to the body at one end of the stent. Transitional portion 445 of the stent between the straight body and the flange may be considered as the “neck” portion. The neck may be gradual (curved, such as in an elliptical shape) or angled (shoulder-like) or otherwise beveled. The stent may have one or more radiopaque markers (not shown) arranged in various positions on the stent for identification of the stent within the patient, such as in transition portion 445 or on the flange itself. The markers are especially important to indicate the transition between the flange and the body for facilitating proper positioning. In a preferred embodiment, a diameter of the flange portion is greater than a diameter of the body portion. Body 440 may have a substantially tubular shape, uniform shape, or tapered shape, among other configurations. Flange 450 may be arranged approximately perpendicular to the axis of the body. The flange can be round or elliptical in shape. Further, the mesh tightness of the body and the flange can be different: e.g., if the body is made with a loose mesh (open cell design) the flange can be made with a tight mesh and vice versa.

FIG. 4B illustrates flange stent 400 from a different angle than FIG. 4A. As illustrated, the flange is an integrated (contiguous) part of the body with a diameter that is larger than that of the body. FIG. 4C illustrates flange stent 400 from a different angle than FIG. 4A. FIG. 4C shows that the flange portion of the stent is substantially perpendicular to the longitudinal axis of the body portion of the stent. FIG. 4D illustrates a top view of flange stent 400 and demonstrates that the flange portion 450 is larger in diameter than the body portion 440 and the mesh tightness of the flange portion is looser than that of the body portion.

In the preferred embodiment, the disclosed flange stent is a self-expanding stent. It may be formed of a plurality of wires that are woven together to form a mesh or grid body for the stent. In one embodiment, the straight portion of the disclosed stent may be formed and be substantially similar to that of a Supera stent. A plurality of wires may be used to form the mesh, such as two, four, six, eight, ten, or twelve or more wires. As is known in the art, the size, diameter, and number of wires may be changed to produce the desired configuration and characteristics of the stent. The mesh tightness can be adjusted according to the specific need, from open cell design to closed cell design, straight or tapered design, and with or without metallic materials. In one embodiment, the mesh density is different between the straight portion of the stent and the flanged portion of the stent. For example, straight portion 440 may be woven with a tighter mesh than flanged portion 450, that is, the obtuse angles between the wire strands are increased. Alternatively, a straight portion can be woven with a looser mesh size, that is the obtuse angles between the wire strands are decreased. These variations in the weave may result in optimal construction of the flange part of the stent. Thus, the mesh tightness, the radial force, the stiffness and the flexibility of the stent can be set in a very wide range, thereby providing a mesh (and resulting stent structure) offers the ideal stent for the lesions in a particular anatomy.

In one embodiment, the self-expanding nature of the disclosed stent allows the stent to expand against the plaque in the artery and exert an outward radial force that resists compression. The greater the density of the mesh (e.g., the more it is a closed cell stent), the greater radial force the stent exerts on the artery. However, excessive radial force can cause stent impaction and plaque protrusion. In some embodiments, a portion of the stent is partially or substantially tapered (instead of substantially uniform in diameter). Tapered designs can be useful when deploying stents across the carotid bifurcation, as the common carotid artery (CCA) is typically larger than the internal carotid artery. The tapered design means that the maximum diameter at the proximal end is larger than that of the distal end, better matching the caliber of the CCA. In one embodiment, tapered stents can have a gradual, conical taper or a more abrupt, shouldered taper.

The disclosed stent may be considered as a bare-metal stent. It may partially or substantially be constructed of nitinol, and may or may not comprise a metal, metal alloy, and/or biodegradable material. For example, combining nitinol with any number of core materials may provide a nitinol composite with significantly increased properties (such as radiopacity, conductivity, or resiliency) while still maintaining its elasticity. Various materials may be paired with nitinol for portions of the disclosed stent, including but not limited to stainless steel, nickel, titanium, gold, platinum, tantalum, palladium, and alloys thereof.

In one embodiment, the disclosed flange stent is formed by using a plain weave technique similar to the weave technique for the Supera stent. In one embodiment, the flange and the body is a contiguous weave such that there is no separation between the wires that make the flange and the body. The flange's shape (e.g., oval, elliptical, angled, shouldered, beveled, etc.) and mesh tightness can be varied according to the needs of the given anatomy. The flange is made with a larger diameter than the body, and the flange is substantially perpendicular to the axis of the cylindrical body. In one embodiment, the transitional portion of the flange is relatively small such that there is a quick transition between the smaller diameter body to the larger diameter flange; this may also occur if the flange diameter is only a little larger than the body diameter. In other embodiments, the transitional portion of the flange is relatively large, such that there is a slow transition between the smaller diameter body to the larger diameter flange; this may also occur if the flange diameter is substantially larger than the body diameter.

There are numerous ways to create the disclosed flange stent, and in particular the flange portion of the stent. Thermal treatment may or may not be used. In one embodiment, the flange portion of the stent may be an integrated part of the stent. The flange portion and body portion may be made of the same wires.

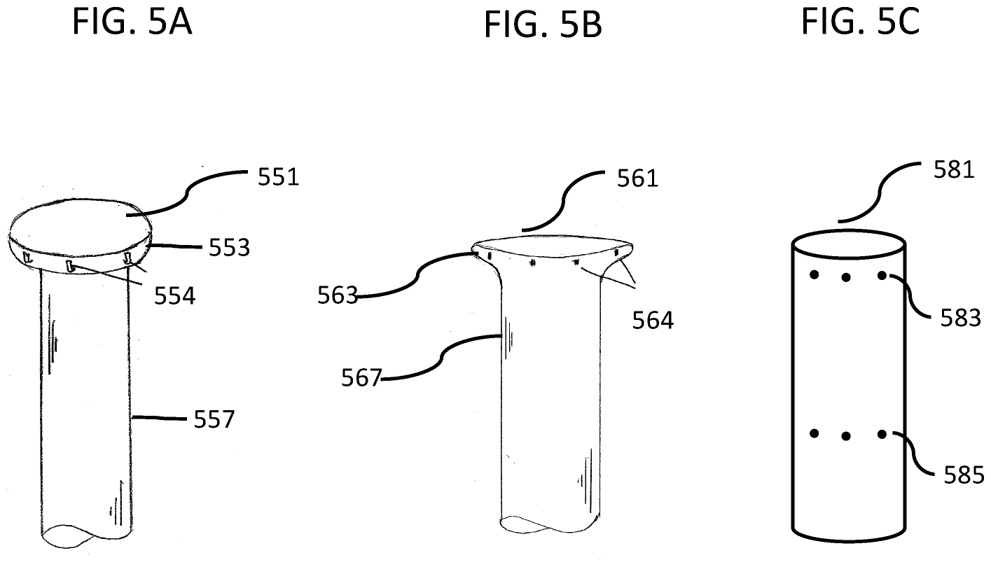

FIGS. 5A-5C illustrate various templates over which the disclosed flange stent may be formed. In a first embodiment, a weave is performed on an appropriately designed template (e.g., jig), such as a template as depicted in FIGS. 5A and 5B. For example, FIG. 5A illustrates template 551 that comprises body 557 and rim 553. Rim 553 may have a plurality of low-profile hooks 554, which are configured to secure the bent wire strands onto the template. Similarly, FIG. 5B illustrates template 561 that comprises body 567 and rim 563. Rim 563 may have a plurality of low-profile hooks 564, which are configured to secure the bent wire strands onto the template. In one embodiment, template 561 is different than template 551 based on the rim and/or transition from the body to the rim. For example, unlike the template in FIG. 5A where the body and the flange are separated from each other with a sharp, shoulder like transition, FIG. 5B illustrates a template that has a smooth, continuously expanding shape on the transition between the body and the rim. The smoother transition allows a more natural shape/configuration of the flange to be formed.

In a first embodiment, weaving of the wire strands of the stent takes place over the appropriate template followed by a heat treatment that programs the final shape. In one embodiment, flange stent 400 may be created from a straight woven stent by creating a flange portion at one of its ends. In one embodiment, the stent is started at the end of the straight stent that does not have welded wire struts. Thus, flange 450 may be created on the end of a substantially straight stent that does not have the back-weave and the unified welded ends of the wire struts.

In a second embodiment, a straight stent portion is woven first. It may be positioned (e.g., constrained) on an appropriately formed template (such as one similar to the template described in FIGS. 5A or 5B) and the stent (or just the end of the stent that is formed into the flange) is heat treated to mold it into the position of the template. In some embodiments, the straight stent portion is heated and then cooled prior to constraining the stent around a flange template for construction of the flange.

In a third embodiment, a substantially straight template (mandrel) may be used, such as one depicted in FIG. 5C. Template 581 may be substantially straight and comprise a first end and a second end. The template may be in the shape of a cylinder. Portions of the template may have low-profile hooks or openings 583, 585, which are configured to secure the bent wire strands onto the template. The hooks/openings may be located at different lengths along the template, such as first openings 583 at a first longitudinal position and second openings 585 at a second longitudinal position. For template 581, the following method may be utilized to form a flange stent. First, a plurality of strands may be woven over the stent template. Second, the density of the wire strands may be varied longitudinally over the stent template. For example, a portion of the strands between the first end and the second end of the stent template may have an increased weave density, thereby forming a first increased density part of the plurality of strands. Third, the method may include heat treating the substantially straight stent to form a flanged portion of the substantially straight stent. The flanged portion may be the part of the stent that has the increased weave density. Fourth, the method may comprise removing the substantially straight stent from the template to form a flanged portion of the substantially straight stent; in other words, once the stent is removed from the template, the stent may be automatically reconfigured into a flange stent with a first portion that is substantially straight and a second portion that is flanged, similar to the above embodiments.

In one embodiment, the flange portion of the stent is made to be more radiopaque than the body portion of the stent. For example, instead of using only nitinol or similar wires, microtubings may be used that are equipped with a highly radiopaque platinum core. As the weave starts with bending the wire filaments in half in their mid-portion to produce wire pairs, it can be calculated how long the segments of these wire pairs containing the platinum core should be to create the flange. Consequently, the flange can be woven with the nitinol microtubing with the platinum core and the body portion of the stent may be produced without the platinum core. Therefore, the optimal visibility of the flange can be achieved. In still another embodiment, the whole flange stent (e.g., the flange portion and a substantial portion of the body portion) may be woven from platinum cored nitinol microtubings.

### Deployment of Flange Stent

The flange stent may be inserted and deployed in a variety of manners. In one embodiment, the method of insertion and deployment depends on whether the flange stent is being used by itself or as part of a modular flange stent system (e.g., coupled to a separate, non-flanged stent). In one embodiment, deployment of the flange stent may be used as an ostial stent.

Similar to the deployment of a Supera based stent, the disclosed stent may be used with a guidewire and delivery catheter. In one example, the size of the delivery catheter may be a 5F or 6F catheter. Other sizes are possible. In one embodiment, the size of the delivery catheter is 7F. This delivery system has the important feature of repositionability, that is, after releasing a certain length of the stent (e.g., approximately 60-80%), and the operator thinks that the stent will be misplaced if the deployment is continued, the operator can withdraw the stent back to the delivery catheter and start the deployment again from a different and more optimal position.

In one embodiment, the flange stent may be delivered similar to techniques described in U.S. Pat. Nos. 8,876,881 and 9,023,095, each incorporated herein by reference. Balloon dilation may or may not be needed with the disclosed flange stent. For example, because the radial force of the woven design can be set in a way that may be enough to overcome the plaque without predilation, any pre-placement balloon dilation can be eliminated from the procedure.

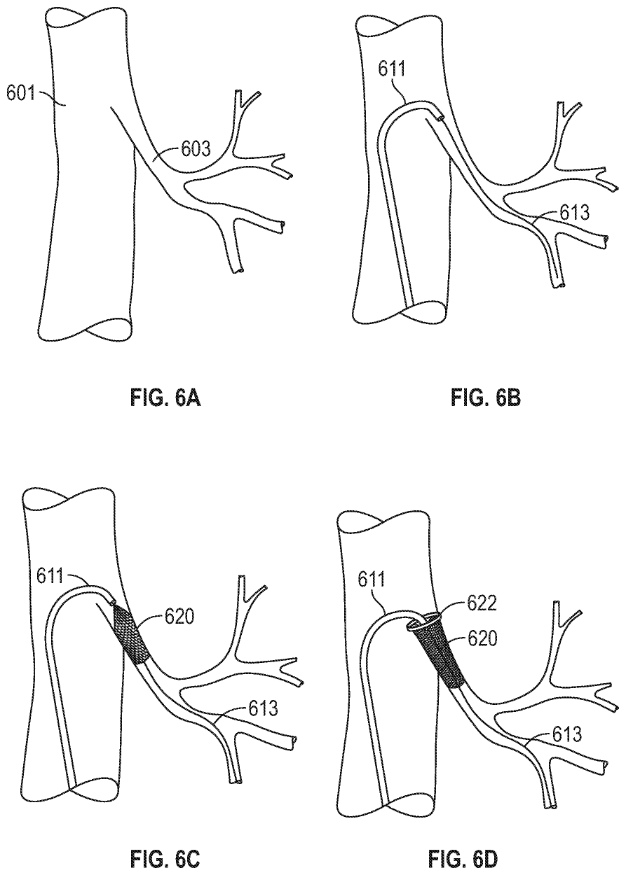

FIGS. 6A-6D illustrate one schematic of deployment of the disclosed flange stent for treatment of an ostial lesion according to one embodiment of the present disclosure. FIG. 6A illustrates one embodiment of abdominal aorta 601 and left renal artery 603. For clarity and to avoid confusion, there is no atherosclerotic plaque, and abdominal aorta 601 and left renal artery 603 are only labelled in FIG. 6A for simplicity purposes.

As shown in FIG. 6B, in one embodiment catheter 611 is advanced into left renal artery 603 over guide wire 613. In this illustration, guidewire 613 is deeply advanced into one of the side branches of the main renal artery to stabilize the system during stent deployment.

As shown in FIG. 6C, flange stent 620 is partially deployed into main renal artery 603 by passing the stent through the catheter. At this point, the operator is watching closely when the highly radiopaque markers (attached to the transition of the body of the stent and the flange portion) are passing out of the tip of the catheter. In one embodiment, once the markers are aligned with the orifice of the renal artery, the operator is ready to deploy the proximal portion of the stent with the flange.

FIG. 6D illustrates a position where flange stent 620 is completely deployed, included the flanged portion 622 of stent 620. As described above, the radiopaque markers attached to the transitional portion of the flange stent (e.g., the neck) serve as a guide for when the flange stent should be released so that the flange stent may be properly positioned. As is known in the art, the operator will have measured the diameter of the vessel and will have sized the stent diameter and length appropriately. Thus, the operator knows where the distal end of the stent should be deployed within the vessel to achieve the proper positioning of the flange of the stent. In one embodiment, proper positioning means that the flange portion 622 of the stent completely covers the area of the main branch adjacent to the orifice, and in some embodiments proper positioning means that the proximal part of the stent (including potentially the flange itself) does not protrude into the lumen of the main branch which may cause partial obstruction of the flow or turbulence, etc.

### Modular Stent

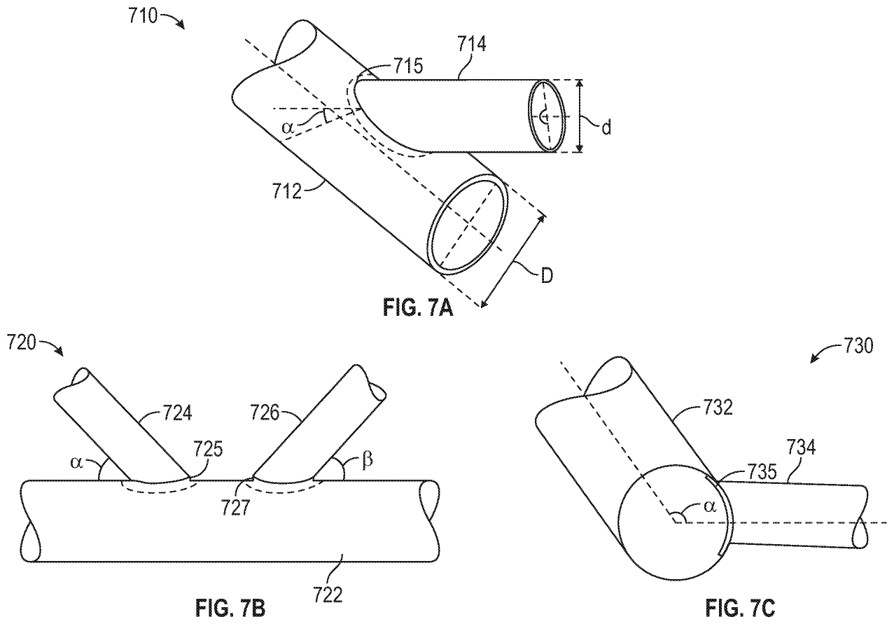

FIGS. 7A-7C illustrate various schematics of a modular stent assembly according to one embodiment of the present disclosure. In one embodiment, disclosed is a modular flange stent that comprises one or more parent stents coupled to a flanged stent. In this embodiment, the flange stent may be used as an independent part of a modular flange stent assembly. In other words, the modular stent assembly may comprise two or more multiple segments of branching/bifurcated anatomy. In one embodiment, a single straight stent may comprise and/or be coupled to one or more flange stents. The modular flange stent may be formed on the templates described in FIGS. 5A-C.

FIG. 7A illustrates one embodiment of a modular stent assembly. As illustrated in FIG. 7A, modular stent 710 consists of a substantially straight stent 712 (e.g., a parent stent) having a diameter “D” and a side branch flange stent 714 having a diameter “d” with flange portion 715 between the side branch flange stent and the parent stent. In one embodiment, diameter D is larger than or equal to diameter d. That is, the larger diameter straight stent is placed into the larger caliber main branch (parent) vessel and the smaller caliber flange stent is placed into the smaller side branch. This scenario is the typical application because of practical reasons—the flange portion adds to the diameter of the flange stent's overall body; therefore, it needs extra space in the parent stent, and the larger caliber parent stent can provide this extra space. Looking at FIG. 7A, side branch flange stent 714 can form an angle alpha (a) with the parent stent. The angle alpha may be acute, right, or obtuse. As illustrated in FIG. 7A, the angle alpha is an acute angle.

FIG. 7B illustrates another embodiment of a modular stent assembly. In this embodiment, a plurality of flange stents is coupled and/or otherwise joined or connected to a parent stent, which in this embodiment is substantially straight. In one embodiment, flange stent 724 and flange stent 726 may join parent vessel stent 722 from opposite directions and at different angles to form modular stent assembly 720. For example, flange stent 724 may form angle alpha (α) with the parent stent and flange stent 726 may form angle beta (β) with the parent stent. In one embodiment, each flange stent comprises a flange portion that connects to the parent stent. For example, first flange stent 724 comprises first flange 725 and second flange stent 726 comprises second flange 727. As in the embodiment of FIG. 7A, the diameter of the parent vessel stent is the same or greater than the diameters of the first and second flange stent.

FIG. 7C illustrates a cross sectional schematic of a modular flange stent assembly 730 at the mating site between the parent stent 732 and the flange stent 734. Flange stent 734 comprises flange portion 735 that connects to parent stent 732. As illustrated, the arch of flange portion 735 is substantially congruent with the inner surface of the parent stent's wall, which provides a preferred arrangement of the flange in the larger caliber patent stent.

The flange stent described herein may be used alone as an ostial stent or be part of a modular stent assembly in which one or more parts of the stent comprises or is coupled to a flanged stent. For example, for branching anatomies, a substantially straight stent (such as a Supera based stent) can be used with one or more of the disclosed flange stents. In one embodiment, a straight Supera (or other self-expanding substantially straight woven stent) based coronary stent is placed in a main branch first, then through the mesh of the main branch stent a flange sent is placed into a side branch. In one embodiment, the flange stent gently pushes away the wires of the main branch stent and when the body of the flange stent is deployed its radial force is able to maintain the lumen of the side branch in spite of the pushed-away wires of the main branch stent. In one embodiment, the flange part of the flange stent mates with the part of the mesh of the main branch stent adjacent to the orifice of the side branch vessel. As a result, the mating site of the straight stent placed in the parent (main) vessel and the flange part of the flange stent will be created at the orifice of the side branch. The wires of the flange will abut to the pushed-away wires of the parent stent and also to the portion of the parent vessel that encircles the orifice. Thus, any bifurcations or branching lesions can be treated, in general, either in the arterial or venous side.

This solution is far superior than existing balloon expanding and self-expanding stents. For example, in a situation where only a straight stent is placed in the diseased main branch, the side branch will be partially occluded by the wires/struts of the stent using either a conventional balloon expanding or a self-expanding stent. The side branch will be “jailed.” The partially obstructed flow into the side branch is problematic even if the side branch is completely intact and not affected by the disease. As described above in the Background Section and in relation to FIGS. 2A-2H, there are numerous disadvantages of using conventional straight stents even in combination with each other. The proposed method and stent disclosed herein can eliminate all the described disadvantages.

For example, the placement of the flange stent through the mesh of the main branch stent gently pushes away the adjacent wires and does not cause any permanent deformation in the stent structure. As another example, the stent coverage by the flange and the main branch stent is contiguous; no gap or non-covered portion is present (in contrast, see FIGS. 2C and 2D which show a gap). Still further, because of the extreme flexibility of the used stents the nature of the covered anatomy is not changed, the stents and the layout of the branches are in harmony, and there is no partial flow obstruction or any change in the flow pattern that could create problems. Further, the disclosed self-expanding stent deployment may not require balloon assistance at all because the radial force is enough to compress the atherosclerotic material to the vessel wall. As another example, the self-expanding properties of the stent helps to ensure that additional luminal gain can be achieved post deployment (days and even weeks after deployment). As another example, the disclosed stent and technique eliminates the potential for spasms caused by elastic recoil due to balloon inflation. Further, the self-expanding nature of the stent also eliminates the tremendous injury that repeat balloon inflations cause.

### Deployment of Modular Flange Stent System

In one embodiment, the disclosed flange stent may be part of a flange stenting modular system, such that it forms one or more components of the modular stent system. In one embodiment, a non-flange stent (such as a substantially straight stent) is inserted first into a first vessel (such as in a main branch) followed by insertion of one or more flanged stents into a second vessel (such as a side branch) through and/or into the straight stent.

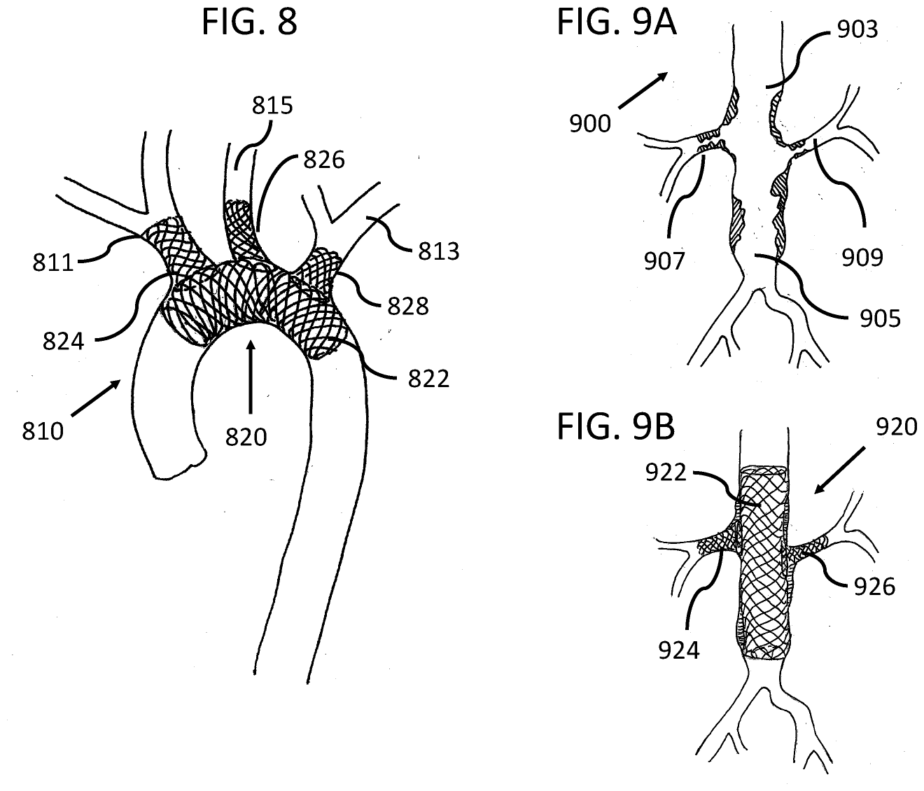

Such a system may be used for treatment of lesions within an aortic arch. FIG. 8 illustrates one schematic of aortic arch 810 with a modular stent assembly according to one embodiment. As is known in the art, aorta arch 810 may comprise various sections, including the three main branches of brachiocephalic artery 811, the left subclavian artery 813, and the left carotid artery 815. As is known in the art, the total length of aortic arch 810, as well as the three main branches, are affected by stenotic atherosclerotic lesions. Minor, moderate, and/or substantial (e.g., 90% or greater) stenosis may exist in any one or more branches or sections of the aorta arch. Prior art stents are not effective on lesions that affect the orifices of the mentioned vessels. Regularly the atherosclerotic lesions of the aortic arch can affect the initial portions of the main vessel, causing ostial stenosis. The standard straight stents cannot adequately address these problems because even if the ideal stent placement was achievable (the proximal edge of the stent is positioned at the level of the orifice), the ostial lesion would not be covered because of its contiguous nature with the adjacent lesions of the main branch. Using straight stents, there are two possibilities. One option is “understenting” the vessel that means that the stent does not cover completely the ostial lesion, therefore, the stenosis here remains unaffected similarly to what is illustrated in FIGS. 1C and 1D. That will result in all the undesired consequences that an untreated stenosis can produce. Understenting occurs when the operator does not want to take the risk of “overstenting,” that is, leaving the proximal portion of the stent protruding into the lumen of the main branch. A second option is overstenting, as seen in FIG. 1B. However, overstenting is equally disadvantageous because the protruding stent can cause turbulent circulation in the main branch and the stents struts (uncovered by neointima) can be a source of thrombus formation, stenosis, embolism.

FIG. 8 illustrates one schematic of aorta arch 810 with modular flange stent 820 deployed in the arch according to one embodiment of the present disclosure. In one embodiment, the disclosed stent 820 has a substantially straight woven stent coupled together with three flange stents. For example, modular flange stent 820 may comprise a substantially straight parent section 822 with three flange stents 824, 826, and 828 coupled to the parent section 822. In one embodiment, the ostial portions of the three main branches of the arch (brachiocephalic, left carotid, and left subclavian artery) are covered with a flange stent. For example, first flange stent 824 is located within brachiocephalic artery 811, second flange stent 826 is located within left carotid artery 815, and third flange stent 828 is located within left subclavian artery 813, In one embodiment, first substantially straight stent 822 is placed in the main vessel of the aortic arch and then a separate flange stent is placed through the mesh of straight stent 822 into each desired side branch. In this embodiment, the complete remodeling of the aortic arch (which is curved) and its main branches can be accomplished.

The disclosed stent may also be used in abdominal aorta applications. For example, FIG. 9A illustrates one schematic of a diffusively diseased abdominal aorta 900. As is known in the art, the abdominal aorta may comprise various sections, including suprarenal segment 903 and infrarenal segment 905, as well as above iliac bifurcation level. As is known in the art, both renal arteries (right renal artery 907 and left renal artery 909) may be frequently affected by ostial lesions. In serious cases the renal involvements are part of a serious stenosing arterioslerotic abdominal aorta. In these cases, both the diseased aorta and the stenosed renal arteries should be addressed by the treatment simultaneously.

FIG. 9B illustrate one schematic of the abdominal aorta with a modular flange stent deployed in the aorta according to one embodiment of the present disclosure. For simplicity, the segments of the abdominal aorta are not separately labelled in FIG. 9B. In one embodiment, stent 920 is a modular stent and is a substantially straight woven stent 922 coupled together with two flange stents 924, 926 placed into the right and the left renal arteries, respectively. In one embodiment, the ostial portions of the renal arteries are covered with a flange stent and deployed in those sections bilaterally. In one embodiment, the placements of the modular stent assembly may be performed by the following steps. First, the main branch is stented with substantially straight stent 922. In this embodiment, a substantially straight and properly sized stent is deployed into the abdominal aorta to cover the diseased portion of the aorta. In one embodiment this will cover at least 2-4 cm long segments both supra-renally and infra-renally. If the atherosclerotic lesions of the aorta are more extensive these portions will be longer (or much longer). Second, after the aortic stent is deployed, one of the renal arteries is catheterized through the mesh of the stent. Third, a guidewire will be advanced deeply into one of the side branches of the renal artery and over the wire a stent delivery catheter with a properly sized flange stent will be advanced. Fourth, the flange stent will be deployed to create a mating site at the level of the renal orifice between the aortic stent and the side branch flange stent. The same procedure will then be repeated in the contralateral renal artery.

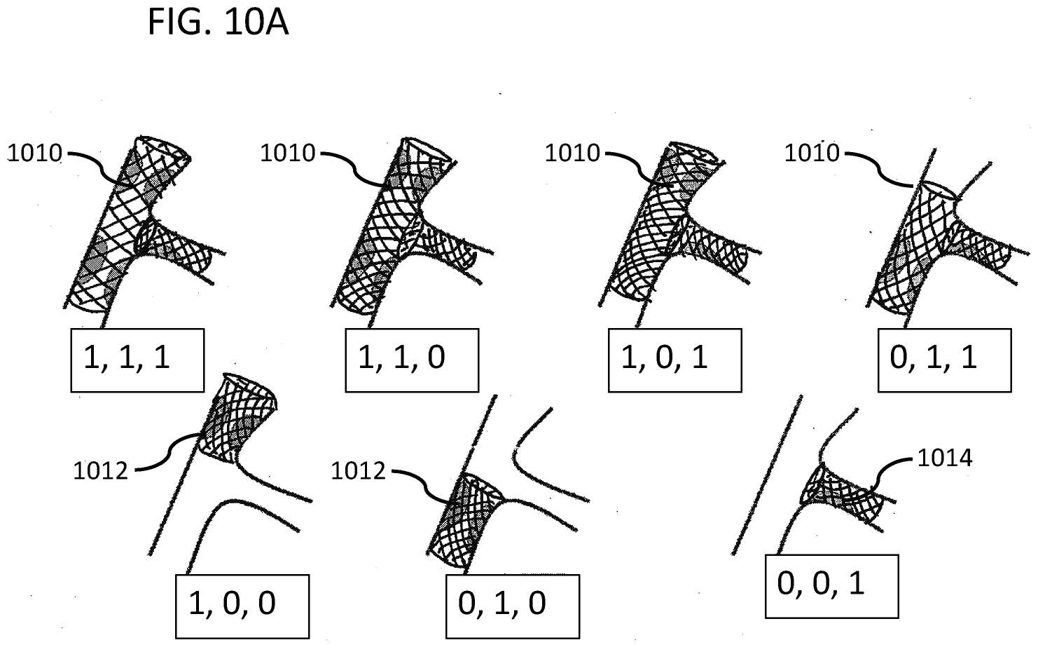

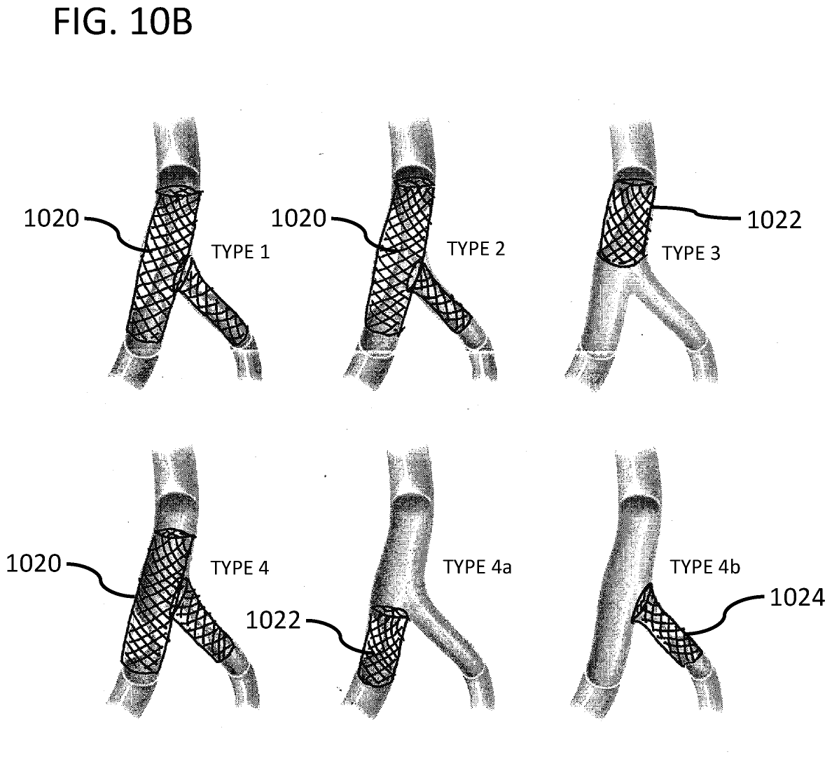

As is known in the art, there are several classifications with respect to the locations of multiple stenosis on the coronary arteries that affect bifurcations. As is known in the art, lesions may exist in any configuration or location of the parent and branch vessels, and may be classified by various mechanisms, such as Types 1-4 of the Lefevre classification or the Medina grading system. Other bifurcation lesion classifications are well known. The disclosed flange stent and modular flange stent offers an excellent solution for a wide variety of ostial lesions. In one embodiment, FIGS. 10A and 10B illustrate how the disclosed modular stent assembly can be used in typical bifurcating lesions on coronary arteries based on standard classifications.

As is known in the art, the Medina classification is based on greater than 50% stenosis in the main branch (proximally or distally to the side branch) and in the side branch itself. FIG. 10A illustrates various stents positioned within the different Medina classifications. It is clear from FIG. 10A that in the majority of the types of lesions a modular stent assembly consisting of a straight stent (parent vessel) and a flange stent (side branch) can be the ideal and/or preferred therapeutic solution. In particular, a modular flange stent assembly 1010 could be used in the 1,1,1; 1,1,0; 1,0,1; and 0,1,1 types of lesions. Indeed, the use of a single straight stent 1012 (e.g., without a flanged portion) may only be optimal for two scenarios (1,0,0 and 0,1,0) and only if the other parts of the bifurcated lesion is substantially intact with no sign of disease. If other parts show definite signs of the stenotic disease (even if the stenosis is only 10-40%, which may or may not be graded appropriately using the Medina classification which typically requires greater than 50% stenosis), the ideal and/or preferred solution would require use of the disclosed modular flange stent assembly. Further, in the case of a 0,0,1 classification (when only the side branch is affected by the disease), a properly sized flange (ostial) stent 1014 offers the optimal solution.

As is known in the art, the Lefevre classification system grades possible therapeutic solutions for the lesions according to Types 1-4. FIG. 10B illustrates various stents positioned within the Lefevre classification. According to the Lefebre classification, lesion types 1, 2 and 4 require a modular stent assembly 1020. For these types, a straight stent is deployed in the main branch and through the mesh of that stent a flange stent is deployed into the side branch. Lesion types 3 and 4a lesions can be treated by straight stents 1022 with precise positioning that does not affect the free flow into the side branch. A Type 4b lesion can be treated with a flange (ostial) stent 1024.

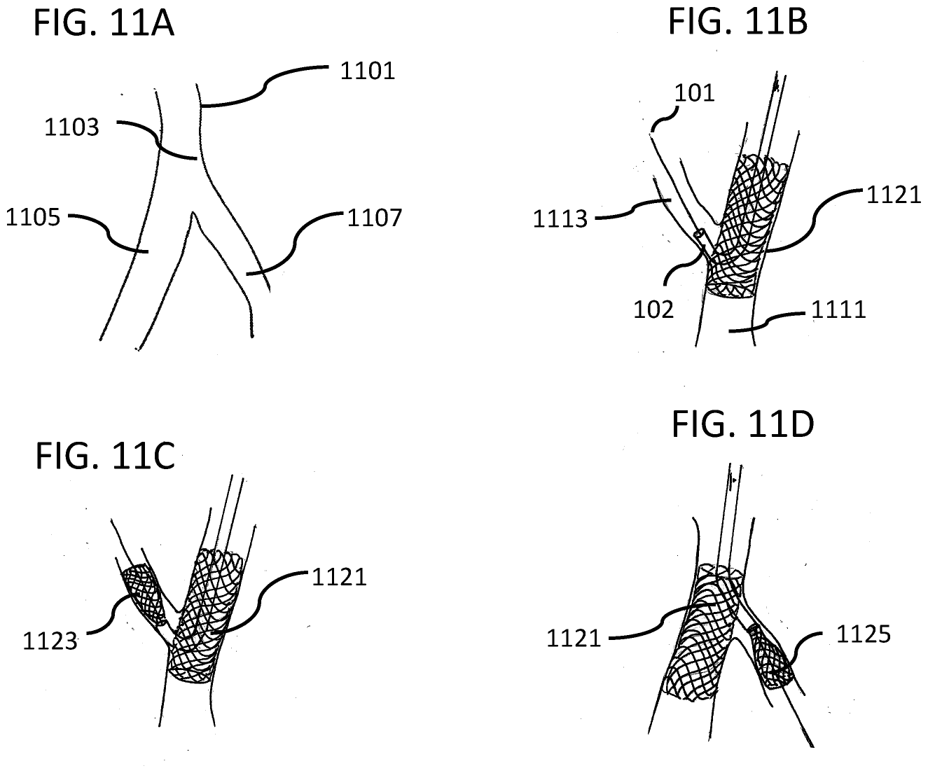

FIGS. 11A-11D illustrate schematics of a branching vessel where ostial stents are deployed in the side branches through the mesh of the stent that was previously deployed in the main branch according to one embodiment of the present disclosure. FIG. 11A illustrates one embodiment of parent vessel 1101 with side branch vessel 1107. For example, the lesion may be located only in the parent vessel only, such as being within pre-branch 1103 only, post branch 1105 only, or both pre-and post-branches 1103, 1105. The lesion may also be located only in the ostial section of the branch, or either pre-branch and ostial or post-branch and ostial.

FIGS. 11B-11D illustrate an operation of installing the disclosed modular flange stent according to one embodiment of the present disclosure. In one embodiment for bifurcation lesions, a first substantially straight stent 1121 is placed in a larger artery 1111 (such as the parent artery or the main branch) and then a separate flange stent 1123 is placed through the mesh of the straight stent into side branch 1113. In some embodiments, if the lesion is located solely in the side branch, then a single flange stent alone may be used to maintain the vessel patency. FIG. 11B illustrates positioning of a substantially straight body stent portion 1121 in a larger artery. As illustrated in FIG. 11B, main branch 1111 is stented with straight woven stent 1121 as is known in the art. Through the mesh of the deployed stent guide wire 101 is first advanced into side branch 1113. Over guide wire 101 the tip of catheter 102 is also advanced through the stent's wire mesh. FIGS. 11C and 11D illustrate positioning a flanged stent portion 1123 or 1125 in a side vessel. As illustrated in FIG. 11C, flange stent 1123 is deployed into the side branch, which is branching off upward in the illustrated figure. FIG. 11D illustrates an alternative embodiment where flange stent 1125 is deployed into a side branch that is at an acute angle with the main branch and is facing downward. In both instances, the straight body portion 1121 is inserted first followed by insertion of the flanged stent portion through a side wall of the main body.

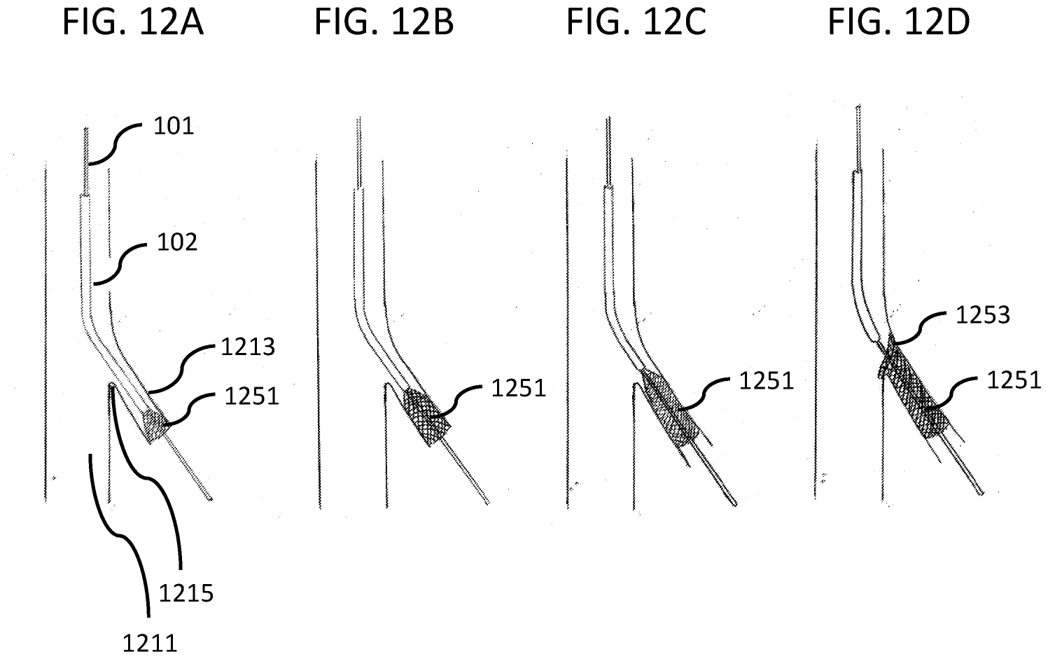

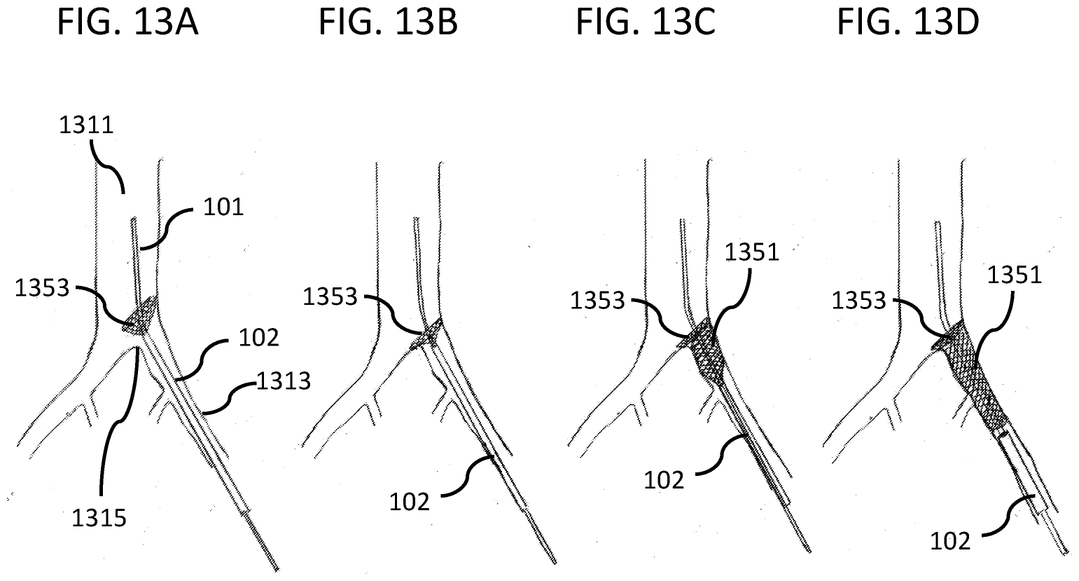

The flange stent may be deployed in a variety of different methods. In one embodiment, the deployment of the flange stent depends on which part of the stent is deployed first. In one embodiment, the flange stent may be deployed from the leading end of the flange or the trailing end of the flange. In one embodiment, the possible access to the given anatomical situation dictates which method should be used. In one embodiment, FIGS. 12A-12D illustrate steps of a trailing end flange deployment of the disclosed flange stent device, while FIGS. 13A-13D illustrate steps of a leading end flange deployment of the disclosed flange stent device. In the “trailing end” deployment method (FIGS. 12A-12D), the body portion of the stent is inserted first followed by insertion of the flanged portion of the stent. The “leading end” deployment method is generally reverse to the trailing end method; the flanged portion of the stent is inserted first followed by insertion of the body portion of the stent. While both methods employ catheter 102 with guide wire 101 to deliver the stent, the loaded delivery catheters differ between the methods only in regards to which end of the stent (the flange or the other end) is close to the tip of the catheter. In one embodiment, the flanged stent utilized in both methods is substantially the same and, similar to the disclosures contained herein, comprises a substantially straight body portion and a flanged portion. In one embodiment, the body portion and flanged portion are formed of a unitary/single construction and inserted as a single piece into the body. In one embodiment, the body stent portion is substantially straight and cylindrical and may comprise a woven nitinol-based stent sized for the particular dimensions of the vessel. The flanged portion may be round or elliptical in shape.

Referring to FIGS. 12A-12D (the trailing end flange deployment method), FIG. 12A illustrates an initial position where a front portion of stent 1251 (e.g., a front portion of the stent body) has been deployed in conjunction with guide wire 101 and catheter 102 in branch 1213 of main vessel or artery 1211. The corner or meeting point of branch and main vessel is labeled as branching section 1215. For simplicity purposes, the guide wire and catheter (as well as the vessel and branch) are not labelled in subsequent figures. While artery 1211 and branch 1213 is shown for illustrative purposes, the disclosed flange stent may be inserted in many other branching (or non-branching) vessels as is obvious to those of skill in the art based on the present disclosure. FIG. 12B illustrates a position where the front half of body stent 1251 is deployed, and FIG. 12C illustrates a position where a substantial portion of the body stent 1251 is deployed. As is known in the art, the stent is deployed by advancing a thumb button connected to a ratchet mechanism that pushes the stent from the delivery catheter in small increments. As illustrated, the end of the stent is adjacent to branching section 1215. FIG. 12D illustrates a last phase of the deployment of the stent according to this embodiment. In this step, flange stent portion 1253 is deployed at the end of straight body stent 1251 and at the corner of branching section 1215. In one embodiment, radiopaque (e.g., platinum) markers are provided at or near the transition of the body of the stent and the flanged portion of the stent. The markers should be aligned with the orifice at the branching section 1215, which is the opening of the side branch at the level of the main branch, before deploying the flanged stent portion at the branch. In another embodiment, the delivery system is constructed with a mechanism for repositionability. During deployment of the flange stent the physician/operator may constantly monitor the position of the already deployed portion of the stent. From the data referring to the stent's nominal size (diameter and length, as well as the distance between front end of the stent body and the flange) as well as previous measurements made by the operator before starting the deployment, the operator will deploy the front end of the stent body accordingly. With built-in repositionability as illustrated in FIG. 12C, the operator still has an additional safety feature. For example, if the operator determines that the final position of the stent will not be correct with special regard to the proper positioning of the flange, the operator can withdraw the portion of the already deployed stent into the delivery catheter and then modify the place of the deployment and restart deploying the stent accordingly.

Referring to FIGS. 13A-13D (the leading end flange deployment method), FIG. 13A illustrates an initial position where a flanged portion 1353 of the stent has been deployed in conjunction with guide wire 101 and catheter 102 in lt. common and external iliac artery 1313 that is a main branch of abdominal aorta 1311. The corner or meeting point of branch and main vessel is labeled as branching section 1315. For simplicity purposes, the guide wire and catheter (as well as the vessel and branch) are not labelled in subsequent figures. While aorta 1311 and branch 1313 is shown for illustrative purposes, the disclosed flange stent may be inserted in many other branching (or non-branching) vessels as is obvious to those of skill in the art based on the present disclosure. In the embodiment illustrated, flange portion 1353 is deployed in the distal portion of abdominal aorta 1311 near aorto-iliac branching section 1315. FIG. 13B illustrates a position where the deployed flange 1353 is pulled back with the delivery catheter 102 as a unit to the level of the orifice and/or branching section 1315 of the left common iliac artery. After flanged stent portion 1353 is fixed in position around the orifice, stent body 1351 may be partially deployed as illustrated in FIG. 13C. FIG. 13D illustrates a last phase of the deployment of the stent according to this embodiment. In this step, the rest of stent body portion 1351 is deployed as catheter 102 is withdrawn along the guide wire. In this leading flange deployment method, the use of radiopaque markers is not of paramount importance, as the flange portion deployed in the lumen of the main branch can be easily visualized. However, in one embodiment, radiopaque markers placed at the transition of the already deployed flange portion and the body of the stent help to confirm the alignment between the flange and the orifice of the side branch vessel during the process when the flange and the delivery catheter as a unit is pulled back to the level of the mating point.

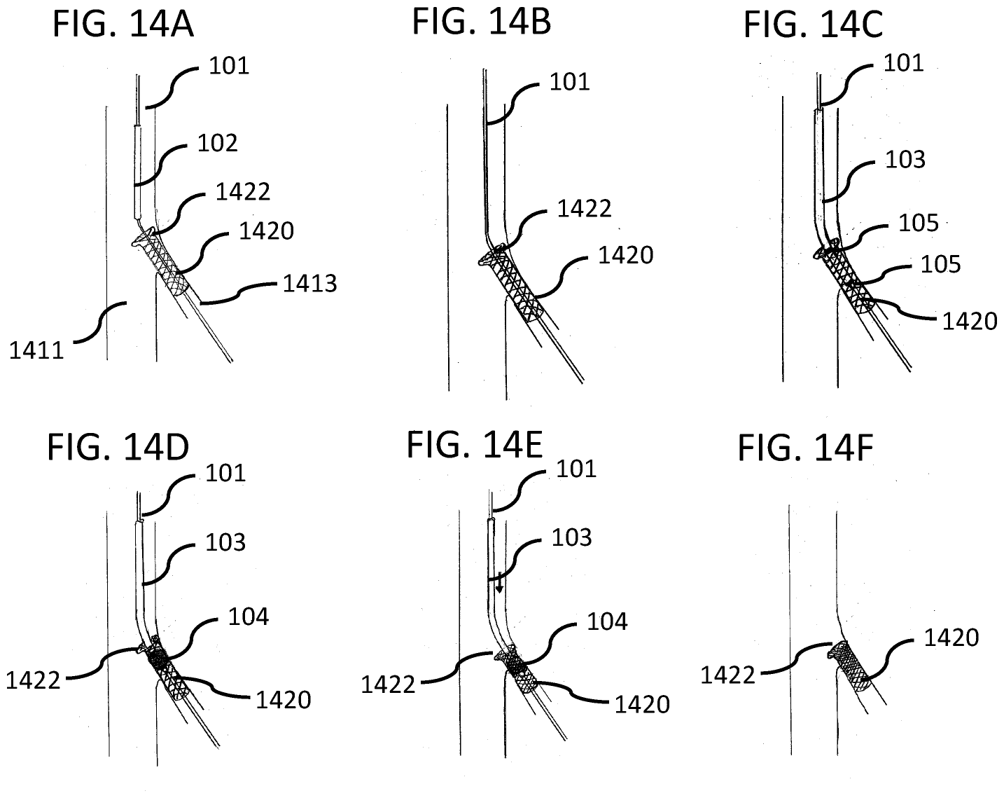

In another embodiment, disclosed is a method for rescuing and/or retrieving a misdeployed flange stent. FIGS. 14A-14F illustrate such a correction procedure. FIG. 14A illustrates a position when flange stent 1420 was misdeployed, which may include flange stent portion 1422 protruding deeply into parent vessel 1411. FIG. 14A illustrates a state of time when the delivery catheter 102 is withdrawn, and guidewire 101 is still in position (which is deeply advanced through the stent within side branch 1413). For simplicity purposes, the main vessel and side branch vessels are not labelled in subsequent figures. FIG. 14B illustrates a position where delivery catheter 102 is removed while leaving guide wire 101 in position. FIG. 14C illustrates a position where balloon catheter 103 is advanced over guide wire 101 and positioned within the initial portion of the flange stent's body 1420. In this illustration, the deflated balloon 104 is between two radiopaque metal markers 105. FIG. 14D illustrates a position where the balloon 104 is inflated to achieve a snug fit with the stent mesh. FIG. 14E illustrates a position in which the catheter 103 with inflated balloon 104 is gently pushed distally over guide wire 101 until the flanged portion 1422 of the stent abuts at the orifice of the side branch. In one embodiment, the stent mesh is stackable, that is, it can be compressed longitudinally. This compression will result in a much tighter mesh in the affected portion of the stent. In one embodiment the compression does not increase the stent diameter. FIG. 14F illustrates a position that shows the stent 1420 successfully corrected and after the balloon catheter and the guidewire are removed. In this embodiment, the proximal portion of the stent mesh is much tighter than that of the distal end.

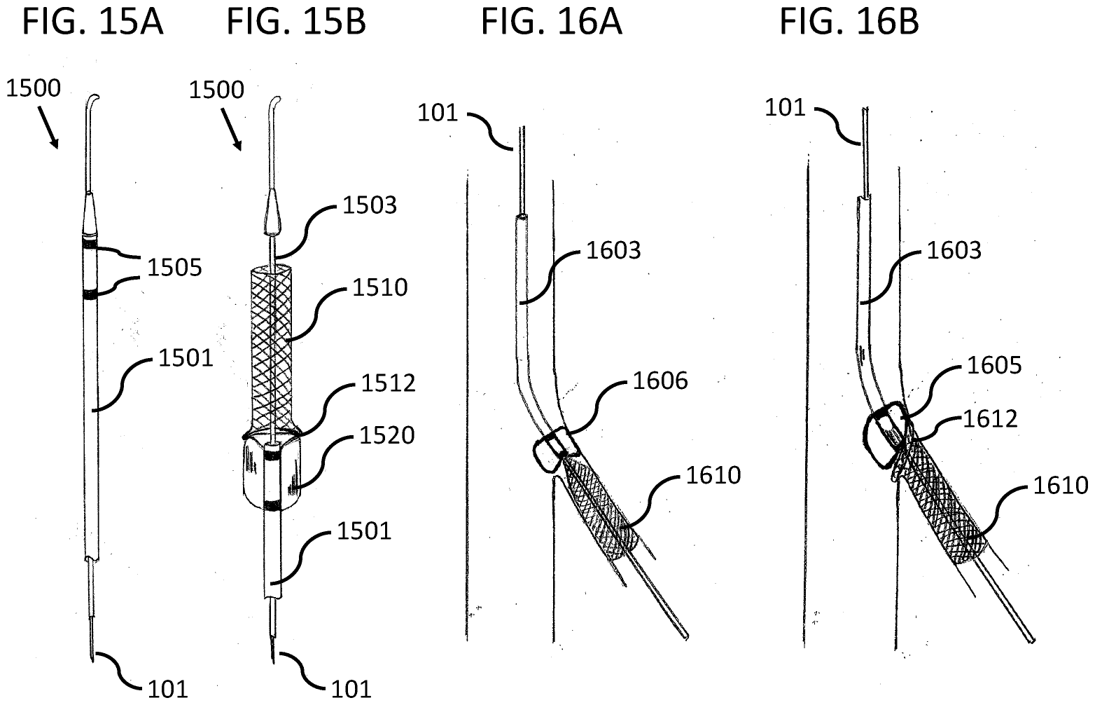

FIGS. 15A and 15B illustrate schematics of a delivery catheter with a stent according to one embodiment of the present disclosure. FIG. 15A illustrates delivery catheter 1500 that comprises an inner catheter (not shown) and outer catheter 1501. As is known in the art, guide wire 101 is advanced within the lumen of the inner catheter. There are two radiopaque (dark) metal bands 1505 on the distal tip of the outer catheter marking the ends of a balloon. FIG. 15B illustrates deployment of balloon 1520 within the delivery catheter of FIG. 15A and shows a position in which outer catheter 1501 is withdrawn thereby exposing inner catheter 1503. Stent 1520 is a flange stent as described above. In this position, flange stent 1520 is in an unconstrained state after deployment and inner catheter 1503 with the guide wire is in the center of the stent. As seen, balloon 1520 is inflated. In one embodiment, flange portion 1512 of the stent and a distal surface of balloon 1520 are congruent and are in an engaged relation with each other.

FIGS. 16A and 16B illustrate schematics of a deployment method for a flange stent according to one embodiment of the present disclosure. In particular, this embodiment illustrates steps of a flange stent deployment with delivery catheter 1603 provided with an assisting balloon 1605, such as that disclosed in FIGS. 15A and 15B. In one embodiment, this method may be used with the trailing flange deployment method as illustrated above in FIGS. 13A-13D. FIG. 16A illustrates a first position where the body of the flange stent 1610 is deployed but not the flange. FIG. 16B illustrates a second position where the flange portion 1612 of the stent has been deployed behind an inflated balloon 1605.

In one embodiment, delivery catheter 1603 is advanced over guidewire 101 close to the target (e.g., a side branch) vessel. The delivery catheter may be further advanced within the side branch vessel to achieve an optimal position for starting the deployment of the distal portion of the body of the flange stent. While the outer catheter is withdrawn the stent is constrained on the inner catheter. The stent body may then be incrementally deployed with the stent driver. As illustrated in FIG. 16A, in one embodiment as the body of flange stent 1610 is completely deployed (which is indicated by radiopaque markers positioned at the transition of the flange and the body of the stent), balloon 1605 of the outer catheter is inflated. Further, inflated balloon 1605 may be positioned in a configuration such that its distal end is aligned with the orifice of the side branch vessel. At this point flange portion 1612 of the stent may be released. In one embodiment, inflated balloon 1605 forces flange 1612 to be deployed in its proper position around the orifice of the side branch as shown in FIG. 16B.

In another embodiment, a balloon on an outer delivery catheter can also be used to correct the position of a mal-deployed flange stent. For example, in one embodiment the delivery catheter with the balloon is advanced into the initial portion of the body of the flange stent (e.g., just behind the flange) and the balloon is inflated. After accomplishing a snug-fit relationship between the balloon and the stent, the stent body is gently moved by pushing the delivery system over the inner catheter/guide wire as a unit distally until the flange abuts around the orifice of the side branch.

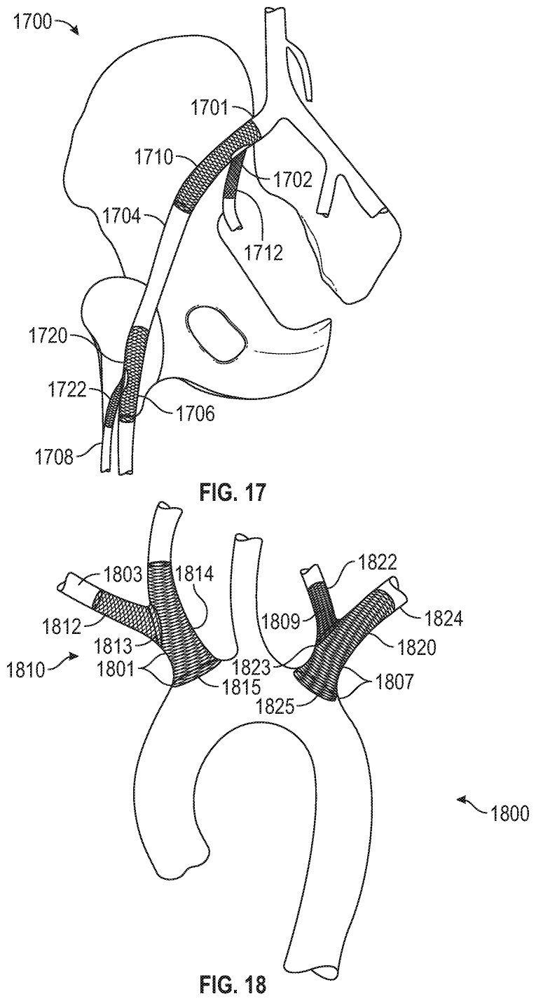

FIG. 17 illustrates one view of the iliofemoral artery system 1700 with two modular flange stent assemblies 1710, 1720 installed therein. As is known in the art, FIG. 17 illustrates the common iliac artery, which bifurcates into internal iliac artery 1702 and external iliac artery 1704. The iliofemoral artery segment is one of the most frequently affected anatomical regions in the case of serious atherosclerosis. Several prior art stents have been and are currently used for treating these lesions, including the Supera based woven nitinol stent. One of the crucial elements of a successful treatment is the preservation of the side branches' patency. In this region the main side branches are the internal iliac artery (hypogastric artery) 1702 and the deep femoral artery 1706 with profunda femoral artery 1708. The patency of these large side branches is very important. The stents placed in the iliofemoral artery can jail these side branches, that is, the stent mesh can partially occlude the orifice of these side branches. The partial occlusion can cause insufficient blood supply in the affected vascular territory, and also significant changes in the flow pattern. Further, the exposed stent mesh that covers the orifice may become a source of thrombosis formation and consequent embolism. To avoid all the disadvantages, properly sized flange stents as disclosed herein can be used to preserve the flow in these branches and avoid any complications. For example, a first flange stent 1710 may be inserted into a portion of the common iliac artery with a flange portion 1712 being located in the internal iliac artery branch. As another example, a second flange stent 1720 may be inserted in the femoral artery 1706 near the junction with the profundal femoral artery, in which will be inserted flange portion 1722 of the stent. Further, the use of flange stent based modular assembly is even more indicated if these side branches themselves are affected by the arteriosclerosis. In these instances, the stenting with the flange stents not only maintains the openings of the vessels but also restore the complete patency of the side branches.

Another possible embodiment of the disclosed modular stent assembly may be when both stents of a modular stent assembly are flange stents. Such a situation is illustrated in FIG. 18. FIG. 18 illustrates aorta arch 1800 with two modular stent assemblies 1810, 1820. Each modular stent assembly comprises two flange stents. For example, first stent assembly 1810 comprises first flange stent 1812 (with flange portion 1813) and second flange stent 1814 (with flange portion 1815); likewise, second stent assembly 1820 comprises first flange stent 1822 (with flange portion 1823) and second flange stent 1824 (with flange portion 1825). In contrast, FIG. 8 illustrates an aorta arch with a single modular stent assembly with a substantially straight stent and three separate flange stents coupled to the straight stent section. A substantially straight or slightly tapered stent 1814 with a trailing flange 1814 is inserted into the rt. brachiocephalic—common carotid artery 1801. The flange addresses the stenotic ostial lesion at the aortic orifice. Through the mesh of stent 1814 another flange stent 1812 is deployed with also a trailing flange 1813 technique in the rt. subclavian artery 1803. FIG. 18 also shows the embodiment where second stent assembly 1820 consists of two flange stents on the lt. subclavian artery 1807. A trailing flange stent 1824 with a substantially straight body is deployed in the lt. subclavian artery 1807. That flange portion 1825 of the stent can treat the ostial stenosis of the lf. subclavian artery, which is a typical location. Through the mesh of the stent 1824 another flange stent 1822 is deployed with the trailing method in the initial portion of the lt. vertebral artery 1809. The latter location is a typical place of an ostial vertebral stenosis.

### Arterial System

As described herein, the disclosed flange stent may be used in numerous applications for the arterial system. It may be used for the treatment of coronary artery diseases (CAD), such as for bifurcation lesions, ectatic vessels and saphenous vein grafts, intervention in the left main coronary artery, intermediate coronary lesions, utility in acute myocardial infarction, and patients with small vessel disease. In general, the disclosed stent is suitable for interventions in various complex coronary lesions, including vein graft lesions, bifurcation lesions, tortuous vessels, left main disease, small vessels, and lesions causing STEMI.

The disclosed stent may be used for carotid arteries, such as an extracranial stent (carotid). Carotid endarterectomy (CEA) decreases the risk of stroke in patients with severe stenosis, although this technique carries the typical risks of open surgery. While carotid artery stenting (CAS) has been demonstrated to be successful, one problem is delayed strokes caused by particulate debris protruding through the stent after the stenting procedure. Various prior art extracranial stent devices include self-expanding stents such as the Wallstent stent and the Precise stent, among others. The disclosed stent system solves many of the problems with prior CAS stents. In one embodiment, the disclosed carotid stent may be used a woven type stent (such as a Supera based stent) with a very tight mesh to avoid debris breaking off from the plaques. The number and size of the wire strands should be carefully selected to achieve the tight mesh and the proper radial force. For example, a smaller wire diameter and more wires in the mesh can create a real tight mesh that eliminates the need for coverage over the metal scaffold. The cell area can be decreased significantly. In one embodiment, the disclosed stent system can avoid protrusion of plaque through the wire strands, allows for side branch patency, conforms tortuous anatomy, and provides good wall apposition.

In similar embodiments to the carotid design, stents can be created for the subclavian arteries and the brachiocephalic artery. Still further, the carotid/subclavian/brachiocephalic stents can also be created with a flange to treat ostial lesions and/or use these stents for a modular assembly through the mesh of a larger stent that is placed in the aortic arch.

The disclosed stent may be used as vertebral artery stents, such as an intracranial stent. One prior art intracranial stent device is the Wingspan Stent. The Wingspan Stent requires pre-dilation and potentially post-dilation. There were multiple problems with the Wingspan Stent, including a problematical “open cell” design, inadequate radial force, the requirement of pre-dilation and optional post-dilation, and the necessity of extensive guidewire manipulation. In one embodiment, the disclosed stent system may be used a woven type stent (such as a Supera based woven nitinol stent) with a mesh that matches the vessel's characteristics and solves the problems presented by the Wingspan Stent. A variation of a vertebral stent can be made with a flange and used as a valid therapeutic solution for the typical vertebral ostial stenosis.

### Venous System

The disclosed stent may also be used for stenting applications in the venous system. In one embodiment, the disclosed stent is substantially the same for both the arterial system and the venous system, although one of skill in the art will recognize that different configurations will be necessary for each vein and/or artery application. In one embodiment, the disclosed venous flange stent (and/or modular system thereof) comprises a woven nitinol stent structure similar to the SUPERA stent. In one embodiment, like the flange stents disclosed above, the disclosed venous flange stent comprises a body portion and a flange portion.

Relative to the arterial system, the venous system is characterized by low pressure, low velocity, large volume, and low resistance. Stenting in the venous system has evolved from experiences in arterial settings. The pathophysiology of arterial stenosis and venous obstruction, however, are very different. Caution must be taken when applying principles of atherosclerotic disease to treatment of venous obstruction related to external compression, mural fibrosis, and intra-luminal webs. In treatment of arterial stenosis, the goal is to restore peripheral perfusion without dissection or extravasation. To avoid these complications, arterial revascularization is performed without the strict perfectionist goal of recreating normal anatomical size of the vessel and often under-correction still achieves acceptable clinical results. Eliminating peripheral venous hypertension, on the other hand, is the goal of treating venous obstruction. Minor residual venous stenosis can contribute to elevated peripheral venous pressures and residual symptoms. Therefore, the critical stenosis threshold in the venous system is regarded by some as being much smaller than that in the arterial system. Stressors on the cavo-ilio-femoral venous system are also different than those within the peripheral arterial system. These veins are exposed to the repetitive trauma of arterial pulsations as well as the changing geometry of the pelvis during ambulation. Additionally, there are mechanical stressors at various anatomical locations that may cause compression and resultant intravascular changes such as mural fibrosis and luminal webs. The external stressor points—as constant anatomical angles—include the iliocaval junction, the iliac bifurcation, and the area posterior to the inguinal ligament.

In one embodiment, a venous stent should balance radial force with flexibility. The stent should be strong at the high-pressure points but flexible at the flex points. For example, the pelvis creates an overall S shape (in front of L5, in the promontory area, there is a high flex point of 120 degrees; deeper in the pelvis, it is about 130-140 degrees; and at the level of the inguinal ligament, another one measuring 130-140 degrees). Thus, in one embodiment a venous stent should be able to form that S shape effortlessly. Flexibility allows the stent to conform to the shape of the vein and move with changing pelvic geometry without kinking or significant decrease in cross-sectional area. In one embodiment, the vein dictates the shape of the stent, not vice versa. Current stents designed for the arterial system are frequently quite rigid. Rigidity leads to nonconformity of the stent and straightening of the vein. If one changes the venous anatomy at one location due to stent rigidity, it might backfire at another and cause early or late stent-related patency loss.

In one embodiment, stent strength is an important quality at the stress points. Stent strength may be characterized by (1) radial force, also known as the chronic outward force or the extent to which the stent pushes outwards, (2) crush resistance, characterized by how much the stent can resist a single load, and (3) hoop strength, also known as radial resistive force, characterized by how much circumferential load the stent can resist. Stent strength is not only needed at the anatomical stress points, but also needed to overcome the high elastic recoil resulting from intraluminal webs and intramural fibrotic tissue which may be present within the diseased vein.

In one embodiment, a venous stent size is larger than that needed in most of the arterial system. Appropriate diameter sizing is key to decreasing peripheral venous hypertension. Stents for the cavo-ilio-femoral system need to be available in a variety of sizes ranging from 10-24 mm or larger. In other embodiments, diseased venous segments may span the entire common and external iliac system. Use of stents with a length that is not optimal often requires stent overlap to cover the length of disease. In one embodiment, stent overlap results in rigidity and decrease conformity within the nonlinear geometrically changing pelvic venous system.

In one embodiment, a venous stent should have deployment that is precise and accurate without foreshortening. The features of repositionability and radiopacity of the stent are also important. The radiopaque material would need to have minimal artifact on magnetic resonance and computed tomographic imaging allowing for better follow-up evaluation. Insertion sheath size is not as important in the venous system as in the arterial system; however, like the stent itself, the delivery system should be flexible to allow for precise placement in the nonlinear venous system.

Further, patients with chronic venous disease are relatively younger than those with atherosclerotic disease, therefore a venous stent must have much longer durability, possibly lasting 50 years or longer and has to perform well over that entire time. That requirement is not present for coronary or peripheral artery stents, which are usually in patients for 5-20 years. Standard fatigue tests have been developed accordingly performing only a limited number of cycles. Principally, tests should be performed to represent 50 years of specific stress movements related to the venous anatomy. The material needs to be corrosion resistant, fatigue resistant, and have negligible in-stent restenosis in the long term.

In summary, the main characteristics of a preferred venous system may include any one or more of the following: deployment without foreshortening, repositionability for precise deployment, tapered—ideal conformity for longer segment, good visibility, good flexibility—high level conformability, resistant against compression and high hoop strength, resistant to repetitive forces (e.g., fatigue resistant for cycles equivalent of 50 years or more), resistant to corrosion, resistant to thrombus formation and platelet adherence, MR compatible, have diameters of approximately 10-26 mm and a length of approximately 40-160 mm (or larger and/or longer), reach and retain the target diameter by balloon angioplasty, are able to cover complex anatomy: confluence, branching, bifurcation, and do no obstruct/hinder venous inflow from side branches (avoiding jailing). The disclosed flange stent may have many of these features.

The disclosed flange stent and/or modular system thereof may be used for venous applications as well as arterial applications. Similar to the arterial application discussed above, a nitinol interwoven stent (such as the SUPERA stent) offers unparalleled flexibility. The SUPERA stent (even if it is constrained into a very acute angle) maintains its lumen without any compromise. Thus, it is able to follow any tortuous anatomy, and it is the anatomy that dictates the shape of the stent and not vice versa. This flexibility results in maximal conformability with the given anatomical structure providing the stent with biomimetic properties. A woven nitinol stent is a strong stent that resists compression and has a high hoop strength. It has a high radial force (outward force) (that unparalleled among the self expanding stents) that ensures good wall contact between the stent and the vessel wall. Its crush resistance is excellent as it has been proven in the most challenging anatomy (femoral-popliteal region). The stent is resistant to any kinking. All these characteristics stemming from the fact that the stent has closed wire ends (unlike, e.g., a Wallstent stent). After the stent is deployed, the ends of the stent are strongly anchored in the vessel and as a result (and in part based on the hoop strength), any single load (crush resistance) or circumferential load by constrictive forces (cause by elastic recoil or stenosis) cannot compromise the lumen of the stent. In one embodiment—and in contrast to the Wallstent stent—all of these forces are evenly present between the two ends of the stent.

In one embodiment, based on the arterial version of the nitinol woven stent (SUPERA), the stent should be modified according to the special requirement of the venous system. For example, there are several ways to achieve the ideal radial force/hoop strength for the venous stents. In some instances, however, excellent expansile force is a must (e.g., under the inguinal ligament or the treatment of the May-Turner syndrome). Careful selection and combination of the diameter of the nitinol wire strands, the number of wire strands used for the structure, and angle between the crossing wires offer plenty of possibilities to find the ideal strength. Simultaneously, the stent's other characteristics can also be modified according to the needs of the special application. For example, the woven structure allows for creating a closed cell design or an open cell design and any version in between.

In one embodiment, the disclosed stent provides deployment capabilities without foreshortening. For example, during deployment, the distal part of the woven nitinol stent anchors firmly itself within the given vascular structure. As the deployment continues more and more length of the stent is getting contact to the vessel wall. In one embodiment, the stent is stackable, that is, the subsequent portions of the stent are deployed continuously behind the already deployed ones. Thus, at the very end of the deployment there is no jump when the very end of the stent exits from the delivery catheter.

In one embodiment, the disclosed stent provides repositionability for precise deployment. For example, the precise positioning of the stent is facilitated by the repositionability that the existing 7F delivery system offers.

In one embodiment, the disclosed stent is tapered and provides optimal conformity for longer segments. For example, for the ilio-femoral veins a 14-10 mm in diameter contiguously tapered stent in proximal-distal direction may be desired. For the treatment of the long iliocaval lesion, the stent should be created in a way that its caval segment be produced with a 22-26 mm in diameter while the distal segment (iliocaval transition) be produced with a 16-14 mm in diameter.

In one embodiment, the disclosed stent provides good visibility. For example, the stent should have adequate radiopacity (such as provided by SUPERA stent), and if a higher visibility is needed, nitinol microtubing with a platinum core can also be used. In one embodiment, the disclosed stent is resistant to repetitive forces, such as up to 10,000,000 cycles or more (such as 40,000,000 cycles) of mechanical cycles without failure. In one embodiment, the disclosed stent is resistant to corrosion, thrombus formation, and platelet adherence. For example, electro-polished nitinol stents may be used. In one embodiment, the disclosed stent should be MRI compatible.

In one embodiment, the disclosed stent has a diameter of approximately 10-26 mm and a length of approximately 40-160 mm (or larger and/or longer). To cover all of the venous system, stents with a plurality of different dimensions should be made. In one embodiment, a special stent may be needed to cover the iliofemoral, iliac, and iliocaval segments, as well as the vena cava inferior and superior. In one embodiment, the disclosed stent structure can retain the target diameter of the vein achieved by angioplasty.

In one embodiment, the disclosed stent does not obstruct and/or hinder venous inflow from side branches. This is important to maintain adequate inflow to prevent vein thrombosis. In one embodiment, the use of appropriately sized flange stents, the inflow can be maintained. The patency of the side branches is especially important when the deployed stent jails the side branches. For example, this is the case when a stent placed into the femoral vein that obstruct the free flow from the profunda (deep) femoral vein. A similar situation exists in the iliac vein, when the placed stent hinders the free flow from the internal iliac (hypogastric) vein.

The disclosed stent may also be a modular stent. In one embodiment, the disclosed stent is configured to cover complex anatomy, such as confluence, branching, and bifurcation applications. Like the arterial system, the disclosed modular stent assembly offers unique solutions in the venous system. In one embodiment, the maintenance of the patency of the side branches and helps to ensure adequate inflow through the stented segment. In these instances, through the stent mesh deployed into the larger (patent) vein (e.g., femoral or iliac veins), a flange stent is deployed into the side branch. In another embodiment, the disclosed stent is able to maintain the patency of important large veins (e.g., renal veins) when either a suprarenal or an infrarenal lesion requires stenting that covers the confluence of the renal vein/veins. In this situation, the protection of the renal vein is important and simultaneous adequate inflow is required from the renal veins to maintain the patency of the stented cava.

Further, it is very common that both iliac vein and the adjacent cava are affected by thrombotic lesions. The current solutions are rather cumbersome. For example, bilateral iliac stenting is suboptimal because the stents protruding into the inferior vena cava can produce turbulence, obstructed flow, and consequent thrombosis. Furthermore, current solutions may obstruct the inflow from the internal iliac veins. The covering of iliocaval confluence with stent/stents is also very challenging because of the natural path of the anatomy (S-shaped curve with multiple flex-points and angles). The current solution is to use multiple stents—even with strikingly different structures (e.g., slotted tube self expanding nitinol stents along with a Gianturco Z-stent). These solutions are awkward.

In one embodiment, the use of a flange stent-based modular stent assembly solves these prior art problems by offering a solution for all types of stenoses and occlusions that in harmony with nature. The disclosed modular stent assembly addresses these prior art problems with a simpler and more cost-effective way, and provides a solution to all of the possible types of the venous stenosis/occlusion categories (see, e.g., Types I-IV of the well known IlioCaval Venous Occlusion (IVCO) classification system according to Crowner J, Marston W, Almaida J, 2014).

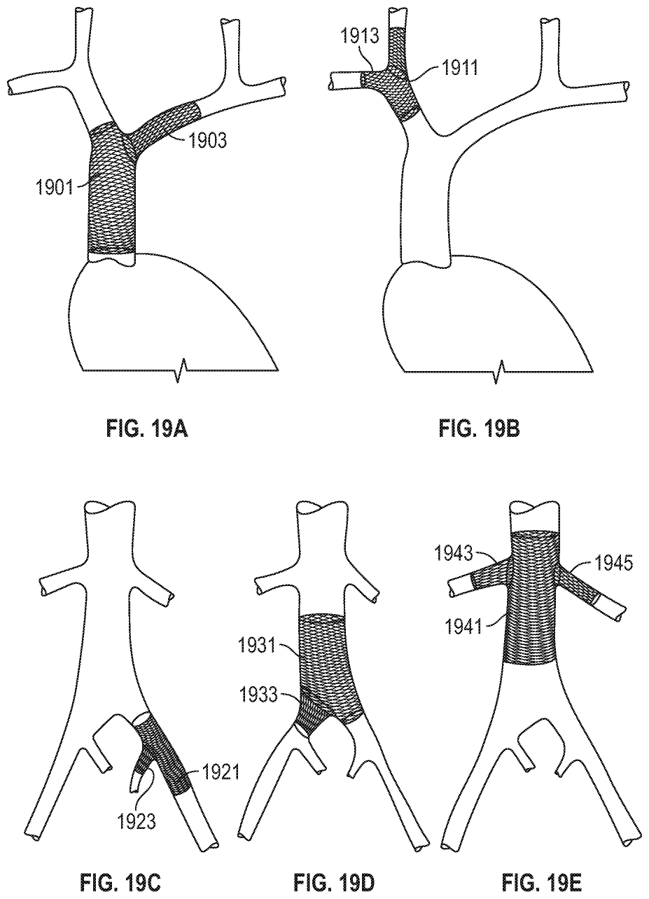

FIGS. 19A-19E illustrate various embodiments of the disclosed flange stent in a variety of venous system applications. FIGS. 19A and 19B show the superior vena cava and related branches. FIGS. 19C-19E show the inferior vena cava and related branches. For simplicity purposes, each of the veins is not labelled, but are clear to one of skill in the art. These flange stents may be deployed in the main and side branches as described above.

FIG. 19A illustrates a substantially straight stent 1901 with a proximal taper positioned in the superior vena cava and the initial portion of the rt. subclavian vein. Straight stent 1901 has a body portion positioned in the superior vena cava and a flanged stent portion 1903 positioned in the lt. brachiocephalic vein.

FIG. 19B illustrates a flange stent positioned in the rt. internal jugular vein through the mesh of a tapered stent body that has already been positioned in the transition between the rt. branchiocephalic vein and the rt. subclavian vein. The flange stent has a body portion 1911 positioned in the rt internal jugular vein and a flanged stent portion 1913 positioned in the rt. subclavian vein.

FIG. 19C illustrates a straight stent positioned in the common and external iliac vein. The flange stent has a body portion 1921 positioned in the external iliac vein and a flange portion 1923 positioned in the internal iliac vein.

FIG. 19D illustrates a substantially straight stent with a distal taper positioned in an infrarenal portion of the inferior vena cava with a small tapered transition located in the lt. common iliac vein. The straight stent has a body portion 1931 positioned in the vena cava inferior. The flange stent 1933 is positioned in the rt. common iliac vein.

FIG. 19E illustrates a straight stent positioned in an infra- and suprarenal vena cava inferior. The straight stent has a body portion 1941 positioned in the vena cava inferior. The flange stents 1943, 1945 and are positioned in the rt. and lt. renal veins, respectively.

All of the methods disclosed and claimed herein can be made and executed without undue experimentation in light of the present disclosure. While the apparatus and methods of this invention have been described in terms of preferred embodiments, it will be apparent to those of skill in the art that variations may be applied to the methods and in the steps or in the sequence of steps of the method described herein without departing from the concept, spirit and scope of the invention. In addition, modifications may be made to the disclosed apparatus and components may be eliminated or substituted for the components described herein where the same or similar results would be achieved. All such similar substitutes and modifications apparent to those skilled in the art are deemed to be within the spirit, scope, and concept of the invention.

Many other variations in the system are within the scope of the invention. For example, the disclosed flange stent may or may not be deployed by itself. In some embodiment, the disclosed flange stent may be an integral part of one or more stenting systems (whether flanged or straight), and thus may be considered a modular flange system. The disclosed flange stent may be used in any anatomical structure and is not limited to branching vessels and can be used to treat ostial lesions. The flange stent may be used in arterial systems and/or venous systems. The disclosed stent may be formed of individual wire strands and/or wire bundles. The wires utilized in the stent may be twisted. The disclosed stent may be formed of a single continuous wire (or wire bundle) or a plurality of wires (or wire bundles). While nitinol may be one shape memory wire used, a variety of other shape memory materials may similarly be utilized. It is emphasized that the foregoing embodiments are only examples of the very many different structural and material configurations that are possible within the scope of the present invention.

Although the invention(s) is/are described herein with reference to specific embodiments, various modifications and changes can be made without departing from the scope of the present invention(s), as presently set forth in the claims below. Accordingly, the specification and figures are to be regarded in an illustrative rather than a restrictive sense, and all such modifications are intended to be included within the scope of the present invention(s). Any benefits, advantages, or solutions to problems that are described herein with regard to specific embodiments are not intended to be construed as a critical, required, or essential feature or element of any or all the claims.

Unless stated otherwise, terms such as “first” and “second” are used to arbitrarily distinguish between the elements such terms describe. Thus, these terms are not necessarily intended to indicate temporal or other prioritization of such elements. The terms “coupled” or “operably coupled” are defined as connected, although not necessarily directly, and not necessarily mechanically. The terms “a” and “an” are defined as one or more unless stated otherwise. The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a system, device, or apparatus that “comprises,” “has,” “includes” or “contains” one or more elements possesses those one or more elements but is not limited to possessing only those one or more elements. Similarly, a method or process that “comprises,” “has,” “includes” or “contains” one or more operations possesses those one or more operations but is not limited to possessing only those one or more operations.

What is claimed is:

1. A self-expanding stent for treating ostial lesions, comprising:
a body with a first end and a second end; and
a flange at either the first end or the second end of the body, wherein the flange is approximately perpendicular to the axis of the body.

2. The stent of claim 1, wherein a diameter of the flange is greater than a diameter of the body.

3. The stent of claim 1, wherein the flange comprises a rim.

4. The stent of claim 1, wherein the flange comprises a round flange.

5. The stent of claim 1, wherein the flange comprises an elliptical shape.

6. The stent of claim 1, wherein the stent comprises a curved transition portion between the flange and the body.

7. The stent of claim 1, wherein the body has a tubular shape.

8. The stent of claim 1, wherein the body has a substantially uniform diameter.

9. The stent of claim 1, wherein the body has a substantially tapered shape.

10. The stent of claim 1, wherein the body and flange are contiguous.

11. The stent of claim 1, wherein the body and flange are made of the same material.

12. The stent of claim 1, wherein the stent comprises a plurality of shape memory wires woven together.

13. The stent of claim 12, wherein the plurality of shape memory wires comprises microtubing.

14. The stent of claim 13, wherein the shape memory microtubing is located on the flange.

15. The stent of claim 1, comprising one or more radiopaque markers proximate to the flange.

16. The stent of claim 15, wherein the one or more radiopaque markers is attached to a transition portion between the flange and the body.

17. The stent of claim 1, wherein the stent is configured to be deployed in an artery or vein.

18. The stent of claim 1, wherein the flange comprises a tighter mesh than the body.

19. The stent of claim 1, wherein the flange comprises a looser mesh than the body.

20. A modular stent system, comprising:
a first stent that is substantially straight or tapered; and
a flange stent coupled to the first stent, wherein the second stent comprises
a body with a first end and a second end, and
a flange at either the first end or the second end of the body, wherein the flange is approximately perpendicular to the axis of the body.

21. The system of claim 20, wherein the first stent and the second stent are configured to be coupled together inside the anatomical body.

22. The system of claim 20, wherein the first stent is configured to be deployed prior to the second stent.

23. The system of claim 20, wherein the second stent is configured to be deployed through a mesh of the first stent.

24. The system of claim 20, wherein the first stent is configured to be positioned in a first vessel and the second stent is configured to be positioned in a second vessel, wherein the second vessel is a side branch of the first vessel.

25. The system of claim 24, wherein the second stent is configured to maintain the patency of the side branch.

26. The system of claim 24, wherein the first vessel is a parent artery or vein.

27. The system of claim 24, wherein the first vessel is a coronary artery.

28. The system of claim 24, wherein the first vessel and the second vessel form a bifurcation, wherein a diameter of the second vessel is less than a diameter of the first vessel.

29. The system of claim 20, further comprising a third stent coupled to the first stent that comprises a flanged body, wherein
the first stent is configured to be positioned in a first vessel,
the second stent is configured to be positioned in a second vessel, wherein the second vessel is a first side branch of the first vessel, and
the third stent is configured to be positioned in a third vessel, wherein the third vessel is a second side branch of the first vessel.

30. The system of claim 20, wherein the system is configured to treat ostial lesions.

31. The system of claim 20, wherein the system is configured to treat a bifurcation lesion.

32. The system of claim 20, wherein the system is configured as a coronary artery stent system.

33. The system of claim 20, wherein the first stent is configured to be positioned in an abdominal aorta and the second stent is configured to be positioned in a renal artery or visceral artery.

34. The system of claim 20,
wherein the first stent is configured to be positioned in an aortic arch and the second stent is configured to be positioned in a brachiocephalic artery, left carotid artery, or left subclavian artery.

35. The system of claim 20,
wherein the first stent is configured to be positioned in a first common iliac artery proximate to an aorto-iliac transition of the common iliac artery and the second stent is configured to be positioned in an opposite second common iliac artery.

36. The system of claim 20,
wherein the first stent is configured to be positioned in an external iliac artery and the second stent is configured to be positioned in an ipsilateral internal iliac artery.

37. The system of claim 20,
wherein the first stent is configured to be positioned in common femoral artery and the second stent is configured to be positioned in an ipsilateral deep femoral artery.

38. The system of claim 20,
wherein the first stent is configured to be positioned in an inferior vena cava—ipsilateral common iliac vein and the second stent is configured to be positioned in the contralateral common iliac vein.

39. The system of claim 20,
wherein the first stent is configured to be positioned in a right brachiocephalic vein and the second stent is configured to be positioned in the ipsilateral right jugular vein.

40. The system of claim 20, wherein the system is configured as a vein stent system.

41. A method of deploying a flange stent, comprising:
inserting a first stent into a first vessel, wherein the first stent comprises a body portion that is substantially straight or tapered;
deploying a flange stent through a side wall of the first stent, wherein the flange stent comprises a body coupled to a flanged end; and
positioning the flange stent proximate to a branching portion of the first vessel.

42. The method of claim 41, wherein the diameter of the flange stent is the same or less than the diameter of the first stent.

43. The method of claim 41, further comprising expanding the flange stent to contact walls of the branching portion of the first vessel.

44. The method of claim 41, further comprising inserting the flanged end of the flange stent through the first stent before the body of the second stent.

45. The method of claim 41, further comprising inserting the body of the flange stent through the first stent before the flanged end.

46. The method of claim 41, further comprising:
deploying a second flange stent through a side wall of the first stent into a second vessel, wherein the second flange stent comprises a body coupled to a flanged end; and
positioning the second flange stent proximate to a branching portion of the second vessel.

47. A method of deploying a flange stent, comprising:
inserting a guide wire into a first vessel;
inserting a catheter with a flange stent over the guide wire, wherein the flange stent comprises a body portion that is substantially straight and a flange portion on one end of the body portion; and
positioning the flange stent proximate to a side branch of the first vessel.

48. The method of claim 47, further comprising expanding the flange stent to contact walls of the side branch.

49. The method of claim 47, further comprising inserting the body portion of the flange stent before the flange portion of the flange stent.

50. The method of claim 47, further comprising inserting the flange portion of the flange stent before the body portion of the flange stent.

51. The method of claim 47, further comprising deploying the flange stent by identification of one or more radiopaque markers attached proximate to the flange portion.

52. The method of claim 47, further comprising inserting a first stent into the first vessel, wherein the first stent comprises a body portion that is substantially straight or tapered.

53. The method of claim 52, wherein the inserting the first stent step is performed before inserting the flange stent, wherein the flange stent is deployed a side wall of the first stent.

54. A method of deploying a flange stent, comprising:
inserting a catheter with a flange stent into a first vessel, wherein the catheter comprises a balloon,
wherein the flange stent comprises a body portion that is substantially straight and a flange portion on a distal end of the body portion;
positioning the body portion within a side branch of the first vessel;
expanding the balloon; and
positioning the flange portion of the flange stent adjacent to an orifice of the side branch by expanding the balloon.

55. The method of claim 54, further comprising expanding the flange stent to contact walls of the side branch of the first vessel with the flange portion of the flange stent.

56. The method of claim 54, further comprising positioning the expanded balloon such that a distal end of the balloon is substantially aligned with an orifice of the side branch.

57. The method of claim 54, further comprising inserting the body portion of the flange stent before the flange portion of the flange stent.

58. The method of claim 54, further comprising deploying the flange stent by identification of one or more radiopaque markers attached proximate to the flange portion.

59. The method of claim 54, further comprising inserting a first stent into the first vessel, wherein the first stent comprises a body portion that is substantially straight or tapered.

60. A method for positioning a misdeployed flange stent, comprising:
advancing a balloon catheter over a guide wire to a first position within a vessel proximate to a flange stent;
positioning the balloon catheter in a first portion of the flange stent;
inflating the balloon catheter to contact an inner surface of the flange stent;
advancing the inflated balloon catheter distally over the guide wire to a second position within the vessel to cause movement of the first portion of the flange stent within the vessel; and
removing the balloon catheter and guide wire.

61. The method of claim 60, wherein the first portion of the flange stent comprises a body portion of the flange stent.

62. The method of claim 60, wherein the second position causes a flange portion of the flange stent to abut an orifice of a side branch of the vessel.

63. The method of claim 60, further comprising compressing a body portion of the flange stent.

64. The method of claim 63, wherein the compressing step creates a tighter mesh in a proximal portion of the flange stent than a distal end of the flange stent.

65. A method of forming a flange stent, comprising:
providing a stent template with a first section that is substantially straight and a second section that comprises a flange;
weaving a plurality of strands around the first section and the second section of the template; and
forming a stent that comprises a body portion and a flange portion with at least some of the plurality of strands.

66. The method of claim 65, wherein the body portion and the flange portion are contiguous.

67. The method of claim 65, wherein the body portion and the flange portion comprise the same plurality of strands.

68. The method of claim 65, wherein the plurality of strands comprises one or more platinum cored microtubings.

69. The method of claim 65, further comprising inserting one or more radiopaque markers at the flange portion of the stent.

70. The method of claim 65, further comprising heat treating the body portion and the flange portion to program the final shape of the stent after the weaving step.

71. The method of claim 70, wherein the heat-treating step comprises heat treating the body portion and the flange portion at approximately the same time for a predetermined heat treatment time.

72. The method of claim 65, wherein the flange portion and body portion are comprised of the same plurality of strands.

73. The method of claim 65, wherein the flange portion is approximately perpendicular to the axis of the body portion.

74. The method of claim 65, wherein the flange portion is woven with a tighter mesh than the body portion.

75. The method of claim 65, wherein the flange portion is woven with a looser mesh than the body portion.

76. A method of forming a flange stent, comprising:
forming a substantially straight stent by weaving together a plurality of strands;
providing a stent template with a first section that is substantially straight and a second section that comprises a flange;
coupling the substantially straight stent to the stent template, wherein a first portion of the stent is coupled to the first section of the template and a second portion of the stent is coupled to the second section of the template; and
heat treating a portion of the substantially straight stent to form a flange from the second portion of the stent.

77. The method of claim 76, further comprising heat treating a portion of the substantially straight stent prior to the coupling step.

78. A method of forming a flange stent, comprising:
forming a substantially straight stent by weaving a plurality of strands over a stent template that is substantially straight, wherein the stent template comprises a first end and a second end;
increasing a weave density of the plurality of strands proximate to one end of the stent template; and
heat treating the plurality of strands woven over the stent template to form a flange portion of the stent.

79. The method of claim 78, further comprising removing the substantially straight stent from the stent template to form a flanged portion of the substantially straight stent.

80. The method of claim 78, wherein the flange portion is woven with a tighter mesh than the body portion.
